(19) United States
(12) Patent Application Publication
(10) Pub. No.: US 2026/0283614 A1
(43) Pub. Date: Sep. 24, 2026

(54) SURGICAL INSTRUMENT

(71) Applicant: LIVSMED INC., Seongnam-si (KR)

(72) Inventor: Woo Jung CHOI, Siheung-si (KR)

(21) Appl. No.: 19/571,407

(22) Filed: Mar. 18, 2026

(30) Foreign Application Priority Data
Mar. 19, 2025 (KR) 10-2025-0035322

(51) Int. Cl. A61B 17/072; A61B 17/00; A61B 90/00

(52) U.S. Cl. CPC A61B 17/07207; A61B 90/06; A61B 2017/00022; A61B 2017/00398; A61B 2017/00862; A61B 2090/064

(57) ABSTRACT

A surgical instrument includes an end tool having one or more jaws, a power transmission part including one or more wires connected to the end tool and configured to transmit a power generated from a power generation part to the end tool, a target member provided in one of the power transmission part and the power generation part, and a sensor part configured to detect a change in the target member according to an operation of the end tool and generate sensing data to obtain information on an operational state of the end tool.

### CROSS-REFERENCE TO RELATED APPLICATION

This application is based on and claims priority under 35 USC § 119 to Korean Patent Application No. 10-2025-0035322, filed on Mar. 19, 2025, in the Korean Intellectual Property Office, the disclosure of which is incorporated by reference herein in its entirety.

### BACKGROUND

### 1. Field

The present disclosure relates to a surgical instrument, and more particularly, to a surgical instrument that is mountable on a robot arm or operable manually, for use in laparoscopic surgery or various other surgical procedures.

### 2. Description of the Related Art

Medically, surgery refers to the treatment of diseases by cutting, incising, or manipulating the skin, mucous membranes, or other tissues using medical devices. In particular, open surgery, which involves incising the skin of a surgical site to treat, shape, or remove an organ therein, may cause bleeding, side effects, patient pain, scars, or the like. Accordingly, alternative procedures such as robot-assisted surgeries or procedures performed by creating a small hole in the skin and inserting only a medical device, for example, laparoscopic surgical instrument, microsurgical microscope, and the like has recently gained significant attention.

A surgical instrument is a tool equipped with an end tool provided on one end of a shaft that passes through a hole formed in the skin, and is manipulated by a medical doctor manually via a predetermined driving part or via a robot arm to perform surgery at the surgical site. The end tool provided on the surgical instrument performs a rotational motion, a gripping motion, a cutting motion, or the like through a predetermined structure.

The background art described above is technical information retained by the present inventors in order to derive the present disclosure or obtained by the present inventors in the process of deriving the present disclosure, and thus is not necessarily known art disclosed to the general public before the filing of the present application.

### SUMMARY

The present disclosure is directed to providing a surgical instrument that is mountable on a robot arm or operable manually for use in laparoscopic or various other surgeries, and that is configured to accurately detect an operation of an end tool and control the end tool to operate within a predefined driving range.

According to an aspect of the present disclosure, a surgical instrument may include an end tool having one or more jaws, a power transmission part including one or more wires connected to the end tool and configured to transmit a power generated from a power generation part to the end tool, a target member provided in one of the power transmission part and the power generation part, and a sensor part configured to detect a change in the target member according to an operation of the end tool and generate sensing data to obtain information on an operational state of the end tool.

In another embodiment of the present disclosure, the target member may support a portion of the one or more wires, and the surgical instrument may further include a control part configured to derive tension information of the one or more wires based on the sensing data related to the change in the target member.

In the other embodiment of the present disclosure, the sensor part may be configured to obtain the sensing data by detecting a force applied to the target member by the one or more wires.

In the other embodiment of the present disclosure, the sensor part may be configured to obtain the sensing data by detecting a movement or a deformation of the target member caused by a force applied by the one or more wires.

In the other embodiment of the present disclosure, the sensor part may be disposed between the target member and a support member that supports the target member, and configured to detect a force applied by the target member to the support member or a relative movement of the target member with respect to the support member.

In the other embodiment of the present disclosure, the sensor part may include a switch member disposed between the target member and the support member and configured to operate according to a movement or a deformation of the target member.

In the other embodiment of the present disclosure, the sensor part may further include a connection member disposed between the target member and the support member to be elastically deformable, and the switch member may be further configured to operate by receiving an external force when the connection member elastically deforms by at least a predetermined amount during the relative movement of the target member with respect to the support member.

In the other embodiment of the present disclosure, the sensor part may be configured to detect a damage of the switch member when a stress greater than or equal to a preset value is applied to the switch member during the movement or the deformation of the target member.

In the other embodiment of the present disclosure, the sensor part may be configured to detect contact between the target member and the support member during the relative movement of the target member with respect to the support member.

In the other embodiment of the present disclosure, the power transmission part may include a drive pulley around which the one or more wires are wound, and which is configured to rotate around an axis of the drive pulley by receiving the power generated from the power generation part, and a pulley frame connected to the power generation part and configured to support the drive pulley.

In the other embodiment of the present disclosure, the power transmission part may further include an auxiliary pulley disposed inside the pulley frame and configured to guide a path of the one or more wires, and an auxiliary pulley support member configured to support the auxiliary pulley, the target member may be the auxiliary pulley, and the sensor part may be disposed between the pulley frame and the auxiliary pulley support member and configured to detect a change in the auxiliary pulley.

In the other embodiment of the present disclosure, the sensor part may be configured to obtain the sensing data by detecting a force applied by the auxiliary pulley support member to the pulley frame.

In the other embodiment of the present disclosure, the sensor part may be configured to obtain the sensing data by detecting a movement or a deformation of the auxiliary pulley support member relative to the pulley frame.

In the other embodiment of the present disclosure, the drive pulley may have one end inserted into a drive pulley coupling part of the pulley frame, the target member may be the drive pulley, and the sensor part may be disposed between the drive pulley and the drive pulley coupling part and configured to detect a change in the drive pulley.

In the other embodiment of the present disclosure, the sensor part may be configured to obtain the sensing data by measuring a force applied by the drive pulley to the pulley frame.

In the other embodiment of the present disclosure, the sensor part may be configured to obtain the sensing data by sensing a movement or a deformation of the drive pulley relative to the pulley frame.

In the other embodiment of the present disclosure, the power transmission part may include a power transmission unit configured to move the one or more wires by a driving of a motor included in the power generation part, and the sensor part may be configured to detect a change in the target member that supports the power transmission unit.

In the other embodiment of the present disclosure, the power transmission unit may include a lead screw configured to rotate by the driving of the motor, having threads, and having one end coupled to a support plate, and a linear movement guide connected to the one or more wires, threadedly engaged with the lead screw, and configured to linearly move according to a rotation of the lead screw, the target member may be the lead screw, and the sensor part may be configured to obtain the sensing data by measuring a force transmitted to the lead screw by the one or more wires.

In the other embodiment of the present disclosure, the sensor part may be configured to detect a force applied to the support plate to which the lead screw is coupled, and measure a force transmitted to the lead screw by a linear movement of the one or more wires.

In the other embodiment of the present disclosure, the surgical instrument may further include a control part configured to derive the information on the operational state of the end tool based on the sensing data obtained by the sensor part, and control the end tool to operate within a preset driving condition.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings.

FIG. 1 is a perspective view illustrating a surgical instrument according to an embodiment of the present disclosure.

FIG. 2 is a perspective view illustrating the remaining parts of the surgical instrument of FIG. 1 except for a manipulation part.

FIG. 3 is a configuration diagram illustrating some components of the surgical instrument of FIG. 1.

FIG. 4 is a schematic perspective view illustrating an end tool of FIG. 1.

FIG. 5 is a perspective view of the end tool of FIG. 4 viewed from another direction.

FIG. 6 is a transparent perspective view of FIG. 4.

FIG. 7 is a perspective view illustrating a first jaw and a cartridge of the end tool of FIG. 4.

FIG. 8 is a perspective view illustrating a power transmission part of FIG. 2.

FIG. 9 is a view illustrating the internal structure of the power transmission part of FIG. 8.

FIG. 10 is a rear view of the power transmission part of FIG. 8.

FIG. 11 is a perspective view illustrating a first pulley frame of the power transmission part of FIG. 8.

FIG. 12 is a perspective view illustrating a second pulley frame of the power transmission part of FIG. 8.

FIG. 13 is a perspective cross-sectional view illustrating a side cross-section of the second pulley frame of FIG. 12.

FIG. 14 is a perspective view illustrating a drive pulley of the power transmission part of FIG. 8.

FIGS. 15 to 17 are views illustrating a process in which a pair of wires are wound around or unwound from the second firing drive pulley of FIG. 9.

FIG. 18 is a perspective view illustrating the power transmission part of FIG. 8 with the first pulley frame removed.

FIG. 19 is a front view of the power transmission part of FIG. 18.

FIG. 20 is a perspective view illustrating wires connected to the drive pulley and a center auxiliary pulley in the power transmission part of FIG. 18.

FIG. 21 is a perspective view illustrating a state in which a wire is wound around an auxiliary pulley in the power transmission part of FIG. 8.

FIG. 22 is a view illustrating the auxiliary pulley positioned in a pulley frame of the power transmission part of FIG. 8.

FIGS. 23 and 24 are perspective views illustrating an auxiliary pulley support member of FIG. 22.

FIG. 25 is a view schematically illustrating a sensor part disposed in the surgical instrument of FIG. 1.

FIG. 26 is a conceptual view illustrating the power transmission part and the sensor part of FIG. 25.

FIG. 27 is a detailed view illustrating the sensor part of FIG. 25.

FIG. 28 is a view illustrating another example of the sensor part disposed in the surgical instrument of FIG. 1.

FIG. 29 is a conceptual view illustrating the power transmission part and a sensor part of FIG. 28.

FIG. 30 is a perspective view illustrating a surgical instrument according to another embodiment of the present disclosure.

FIG. 31 is a perspective view illustrating a manipulation part and a power generation part of the surgical instrument of FIG. 30.

FIG. 32 is a magnified perspective view illustrating the power generation part of FIG. 31.

FIG. 33 is a cross-sectional view illustrating a gear structure of the power generation part of FIG. 31.

FIGS. 34 to 36 are conceptual diagrams illustrating a power transmission structure of the surgical instrument of FIG. 30 and operations of the end tool.

FIG. 37 is a view schematically illustrating a sensor part disposed in the surgical instrument of FIG. 30.

### DETAILED DESCRIPTION

Hereinafter, the following embodiments will be described in detail with reference to the accompanying drawings. When describing with reference to the drawings, identical or corresponding components will be assigned the same reference numerals, and duplicate descriptions thereof will be omitted.

Since various transformations can be made to these embodiments, specific embodiments will be illustrated in the drawings and described in detail in the detailed description. The effects and features of the present embodiments and the accompanying methods for achieving them will become apparent from the following description of the contents, taken in conjunction with the accompanying drawings. However, the present embodiments are not limited to the embodiments disclosed below, but may be implemented in various forms.

In describing the present disclosure, a detailed description of known related arts will be omitted when it is determined that the gist of the present disclosure may be unnecessarily obscured.

In the following embodiments, singular forms are intended to include plural forms as well, unless the context clearly indicates otherwise. Although terms such as “first,” “second,” and the like may be used to describe various components, such components should not be limited to the above terms The terms are only used to distinguish one component from another.

In the following embodiments, terms such as “include” or “have” mean that the features or components described in the specification are present, and the possibility that one or more other features or components will be added is not excluded in advance.

In the following embodiments, when a unit, region, or component is referred to as being formed on another unit, region, or component, it can be directly formed on the other unit, region, or component. That is, for example, intervening units, regions, or components may be present.

In the following embodiments, terms such as “connecting” or “coupling” two members do not necessarily mean a direct and/or fixed connection or coupling of the two members, unless the context clearly indicates otherwise, and do not preclude other members from being interposed between the two members.

Sizes of components in the drawings may be exaggerated or reduced for convenience of description. For example, since the size and thickness of each component shown in the drawings are arbitrarily illustrated for convenience of description, the following embodiments are not necessarily limited thereto.

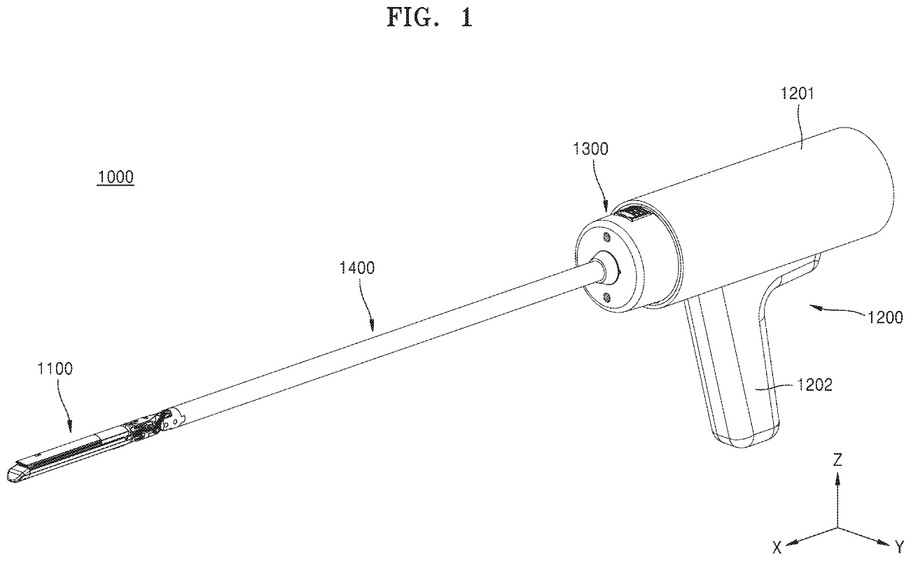

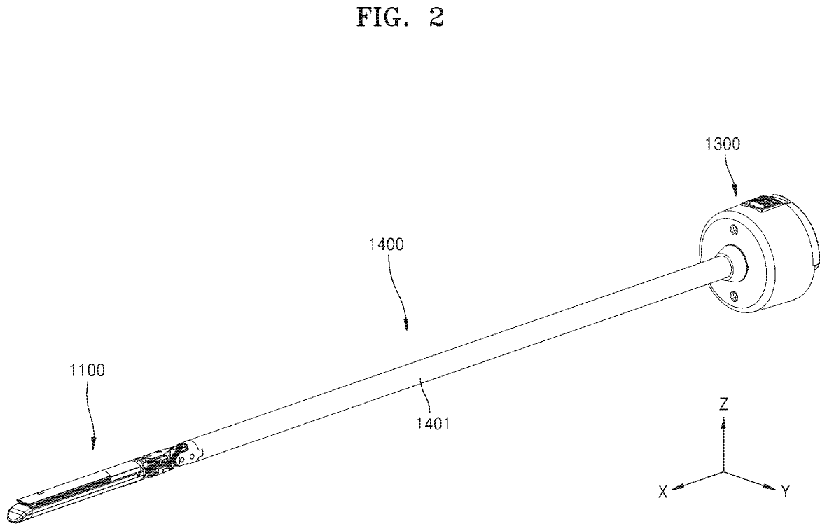

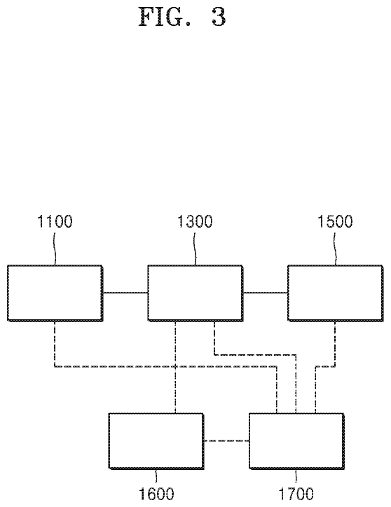

FIG. 1 is a perspective view illustrating a surgical instrument 1000 according to an embodiment of the present disclosure, FIG. 2 is a perspective view illustrating the remaining parts of the surgical instrument 1000 of FIG. 1 excluding a manipulation part 1200, and FIG. 3 is a configuration diagram illustrating certain components of the surgical instrument 1000 of FIG. 1.

Referring to FIGS. 1 to 3, the surgical instrument 1000 may include an end tool 1100, the manipulation part 1200, a power transmission part 1300, a connection part 1400, and a power generation part 1500.

Here, the connection part 1400 is formed in the shape of a hollow shaft, in which one or more wires and electric wires may be accommodated. For convenience of description of the present embodiment, the connection part 1400 will be also referred to and described as the shaft 1400. The manipulation part 1200 is coupled to one end portion of the connection part 1400, the end tool 1100 is coupled to another end portion thereof, and the connection part 1400 may serve to connect the manipulation part 1200 to the end tool 1100. For example, the connection part 1400 may include a straight portion 1401, and although not shown in the drawing, the connection part 1400 may include one or more curved portions for ease of use.

The power transmission part 1300 may be formed on one end portion of the connection part 1400 and may serve to transmit power generated by the power generation part 1500 to the end tool 1100. For example, the power transmission part 1300 may be positioned between the end tool 1100 and the manipulation part 1200. As will be described later, when a user such as a medical doctor manipulates the manipulation part 1200, the power generation part 1500 generates power to control the end tool 1100, and the generated power may be transmitted to the end tool 1100 through the power transmission part 1300. The power transmission part 1300 may include a plurality of wires, a plurality of pulleys, a plurality of links, a plurality of joints, a plurality of gears, and the like. The power transmission part 1300 described above will be described in detail later.

A user may operate the end tool 1100 by manipulating the manipulation part 1200. For example, the manipulation part 1200 is a component enabling the user to input signals to control the motions of the end tool 1100. That is, the manipulation part 1200 may be described as a component that receives signals from the user to control motions of the end tool 1100. Here, the signals for controlling the motions of the end tool 1100 may correspond to mechanical manipulations such as pressing a button or switch, or rotating or moving a particular member, and may also be electrical signals generated by such mechanical manipulations, but the present disclosure is not limited thereto.

The manipulation part 1200 is provided as an interface to be directly controlled by a medical doctor; for example, it may be provided in a gun shape, a tongs shape, a stick shape, a lever shape, or the like, and when the medical doctor controls the manipulation part 1200, the end tool 1100, which is connected to the corresponding interface and inserted into the body of a surgical patient, performs a certain motion, thereby performing surgery. Here, the manipulation part 1200 is illustrated in FIG. 1 as being formed in a gun shape, but the concept of the present disclosure is not limited thereto, and various types of manipulation parts capable of connecting to the end tool 1100 and manipulating the end tool 1100 may be possible.

The manipulation part 1200 may include a housing 1201 forming an outer shape thereof. As will be described later, at least a portion of the power generation part 1500 configured to generate power to control the end tool 1100 may be accommodated inside the housing 1201. Further, a circuit unit for controlling the operation of the power generation part 1500 and a slip ring for supplying electrical energy to the power generation part 1500, connecting communication, or transmitting various other signals may be accommodated inside the housing.

A handle 1202 may be formed on the manipulation part 1200. The handle 1202 is a part for a user to grip. Thus, the user can use the surgical instrument 1000 according to the present disclosure while gripping the handle 1202 of the manipulation part 1200.

For example, although not shown in the drawings, a button, a switch, a lever, and the like for controlling various motions of the end tool 1100 may be further formed in the manipulation part 1200.

The end tool 1100 is formed on another end portion of the connection part 1400, and performs necessary motions for surgery by being inserted into a surgical site. As an example of the end tool 1100, a pair of jaws for performing a grip motion may be used. However, the concept of the present disclosure is not limited thereto, and various devices for performing surgery may be used as the end tool 1100. For example, a configuration such as a cantilever cautery may also be used as the end tool. The end tool 1100 is connected to the manipulation part 1200 by the power transmission part 1300 (e.g., a wire or the like), and receives a driving force of the manipulation part 1200 to perform a motion necessary for surgery, such as gripping, cutting, suturing, or the like.

In describing the present disclosure, the portion closer to a user side, i.e., the portion closer to the manipulation part 1200, will be referred to as a proximal end, and the portion farther from the user side, that is, the portion farther from the manipulation part 1200, will be referred to as a distal end.

When a user manipulates the manipulation part 1200, the power generation part 1500 may generate power to control the end tool 1100 based on the manipulation. The power generation part 1500 may have at least one motor that generates power. The power generated from the power generation part 1500 may be transmitted to the end tool 1100 through the power transmission part 1300, and as a result, the end tool 1100 may operate. A power generation and transmission structure of the surgical instrument 1000 will be described in detail below.

The surgical instrument 1000 may further include a sensor part 1600 and a control part 1700.

The end tool 1100 may rotate by receiving power through the power transmission part 1300 and may perform various motions such as gripping, cutting, and suturing.

For example, the end tool 1100 may receive power and rotate within a predetermined angular range. Alternatively, the end tool 1100 may receive power and allow an operation member to move within a predetermined section. Alternatively, the end tool 1100 may receive power and clamp tissue having a predetermined thickness.

In some embodiments, the operation range of the end tool 1100 may be limited. When the end tool 1100 operates beyond a predetermined range, the surgical instrument 1000 may be damaged or may fail to perform normal surgical operations. Accordingly, driving conditions related to the operation range of the end tool 1100 may be preset, and the end tool 1100 may be controlled to operate within such driving conditions. At this time, the power to be transmitted to the end tool 1100 may also be limited by setting.

For example, when the end tool 1100 rotates excessively or when the operation member of the end tool 1100 moves forward beyond a predetermined driving range, there may be a risk of damage to the surgical instrument 1000, such as breakage of a wire connected to the end tool 1100 or fracture of the jaw. Alternatively, when tissue is excessively thick when the end tool 1100 clamps the tissue for surgery, the end tool 1100 may fail to operate normally.

To prevent component damage caused by excessive operation of the end tool 1100 and to perform surgical operations normally, the surgical instrument 1000 needs to sense an operational state of the end tool 1100 and control the operation of the end tool 1100 based on the sensed state. The sensor part 1600 may generate data to obtain information on the operational state of the end tool 1100.

The sensor part 1600 may detect changes in a predetermined member associated with the operation of the end tool 1100, and at this time, the member that is sensed by the sensor part 1600 is defined as a target member. The sensor part 1600 may obtain sensing data by detecting a movement amount, a deformation amount, or damage of the target member, or a force applied to the target member. The sensing data may be used to determine whether the end tool 1100 operates normally within the preset driving conditions.

The control part 1700 may obtain information on the operational state of the end tool 1100 based on the sensing data generated by the sensor part 1600. The control part 1700 may directly use the sensing data or derive information related to the operational state of the end tool 1100 through predetermined computation or processing.

The information related to the operational state of the end tool 1100, which is obtained by the control part 1700, may include information on power supplied to the end tool 1100. In addition, the information related to the operational state of the end tool 1100, which is obtained by the control part 1700, may include information on the extent of operation, such as a rotation angle of the end tool 1100, a travel distance of the operation member, and a clamping gap. In some embodiments, the control part 1700 may control the end tool 1100 to operate within the preset driving conditions based on the sensing data and the information related to the operational state of the end tool 1100.

Details regarding the generation of sensing data by the sensor part 1600, the process by which the control part 1700 obtains the information related to the operational state of the end tool 1100, and the control of the end tool 1100 based on the obtained information will be described in detail below.

Hereinafter, the end tool 1100 of the surgical instrument 1000 of FIG. 1 will be described in more detail.

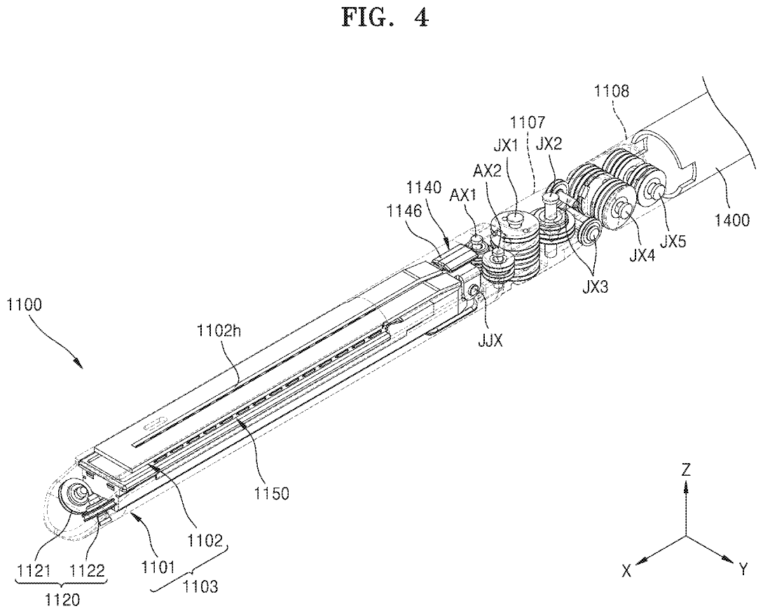

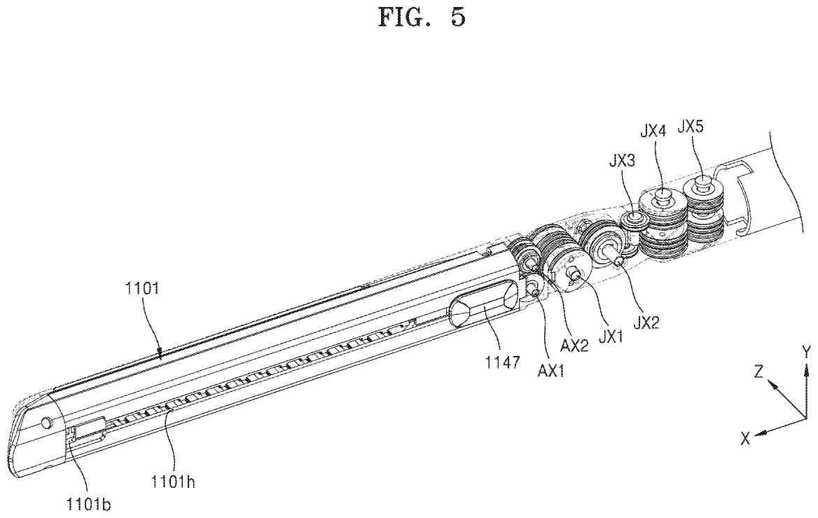

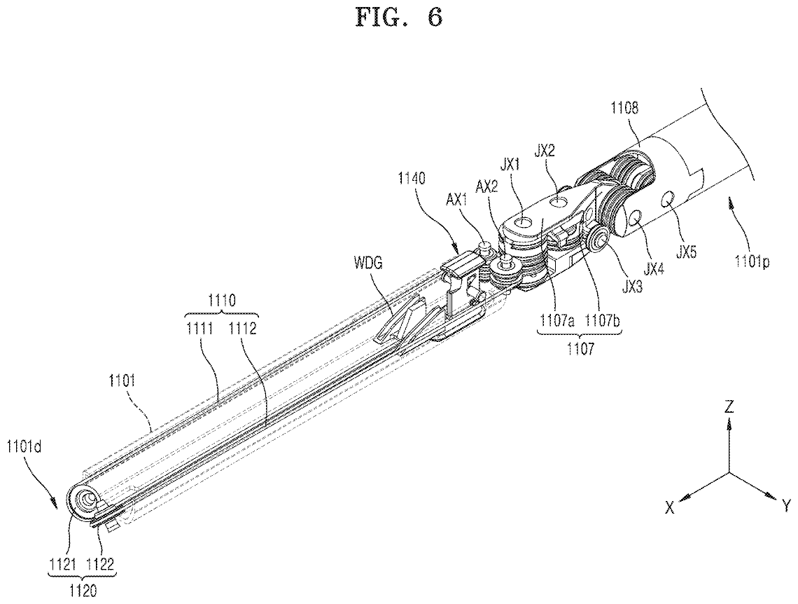

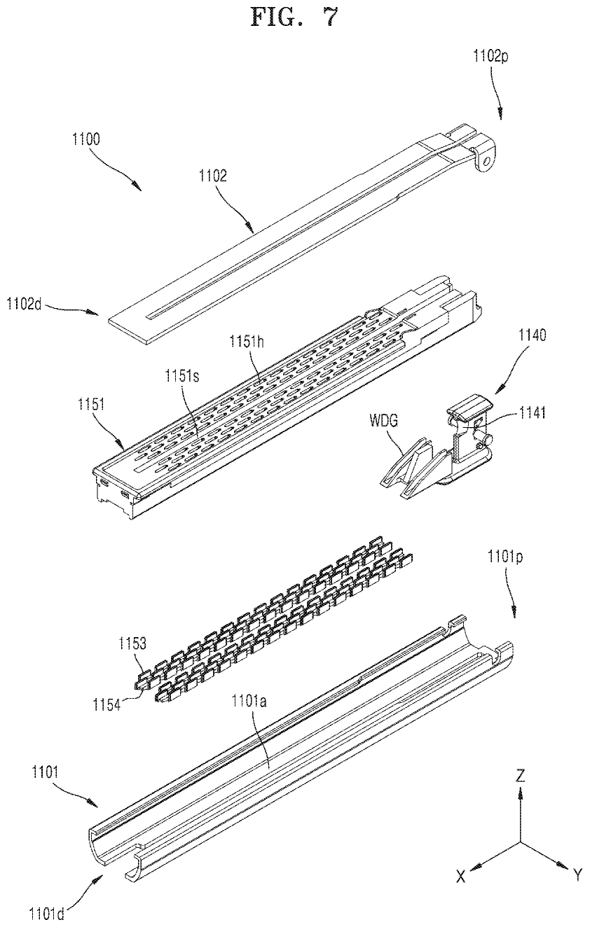

FIG. 4 is a schematic perspective view illustrating the end tool of FIG. 1, and FIG. 5 is a perspective view of the end tool of FIG. 4 viewed from another direction. FIG. 6 is a transparent perspective view of FIG. 4, and FIG. 7 is a perspective view illustrating a first jaw and a cartridge of the end tool of FIG. 4.

Referring to FIGS. 4 to 7, the end tool 1100 may include jaws 1103, a plurality of fixed pulleys 1120, and a plurality of advancing wires 1110. The plurality of fixed pulleys 1120 may have two or more pulleys. For example, the plurality of fixed pulleys 1120 may include a first fixed pulley 1121 and a second fixed pulley 1122. The plurality of advancing wires 1110 may have two or more wires. For example, the plurality of advancing wires 1110 may include a first advancing wire 1111 and a second advancing wire 1112.

The jaws 1103 may perform various functions, such as a grip motion. For example, the jaws 1103 may be provided as a pair and may include a first jaw 1101 and a second jaw 1102. Here, each of the first jaw 1101 and the second jaw 1102, or a component encompassing the first jaw 1101 and the second jaw 1102, may be referred to as the jaw 1103.

A cartridge 1150 may be disposed to be accommodated in the first jaw 1101, and a plurality of staples 1153 may be positioned inside the cartridge 1150. In a state in which the first jaw 1101 and the second jaw 1102 are close to each other, e.g., when body tissue is interposed therebetween and the first jaw 1101 and the second jaw 1102 are in a closed state, the operation member 1140 may receive a force through the plurality of advancing wires 1110. As a result, the operation member 1140 may move toward a distal end 1101d of the first jaw 1101 to push the staples upward and perform stapling.

In this case, one or more clamps 1146 and 1147 of the operation member 1140 may protrude to the outside of the first jaw 1101 and the second jaw 1102, allowing the operation member 1140 to move forward while applying pressure to outer surfaces of the first jaw 1101 and the second jaw 1102, which facilitates the smooth progression of a stapling process.

The cartridge 1150 may include a cover 1151 that accommodates the staples 1153 and a case 1152 corresponding to the bottom, and may be configured in a form in which the case 1152 is positioned on the first jaw 1101. Staple holes 1151h, through which the plurality of staples 1153 may be ejected to the outside, may be formed in the cover 1151. The staples 1153, which are accommodated in the cover 1151 before a stapling operation, may be pushed upward by the operation member 1140 during the stapling operation. As a result, the staples 1153 may pass through the staple holes 1151h of the cover 1151 and may be ejected to the outside of the cartridge 1150, thereby performing stapling.

The operation member 1140 may be used together with a wedge WDG. For example, the wedge WDG may be prepared separately from the operation member 1140 and then positioned adjacent to the operation member 1140 in the first jaw 1101. As another example, the operation member 1140 and the wedge WDG may be integrally formed as one body. The wedge WDG may be positioned on at least one side of a body 1142 of the operation member 1140 and formed to have a predetermined inclined surface. That is, the wedge WDG may be formed to be inclined by a certain degree with respect to a direction in which the end tool 1100 extends. In other words, the wedge WDG may be formed to have a greater height at a proximal end 1101p side of the first jaw 1101 than the distal end 1101d side of the first jaw 1101.

The wedge WDG may be formed to be sequentially in contact with withdrawal members 1154 or the plurality of staples 1153 positioned in the cartridge 1150, and may serve to sequentially push the staples 1153 upward.

In some embodiments, a slit 1151s may be formed in the cover 1151 in a longitudinal direction of the cover 1151. A blade 1141 of the operation member 1140 may protrude to the outside of the cartridge 1150 through the slit 1151s. As the blade 1141 of the operation member 1140 moves along the slit 1151s, the tissue that has been stapled may be cut.

The plurality of fixed pulleys 1120 may be disposed in the first jaw 1101 to be closer to the front of the cartridge 1150, i.e., to the distal end 1101d of the first jaw 1101, than the cartridge 1150. For example, the plurality of fixed pulleys 1120 may be disposed in a front space 1101c of the first jaw 1101, and details thereof will be described below.

In some embodiments, the end tool 1100 of the surgical instrument 1000 of the present embodiment may include one or more members, such as joint members, that connect the jaw 1103 to the connection part 1400. In an optional embodiment, the end tool 1100 may include an end tool hub 1107 and a pitch hub 1108.

The pitch hub 1108 may be disposed to connect the end tool 1100 to the connection part 1400.

For example, a fourth pulley shaft JX4 may correspond to the pitch hub 1108, and the fourth pulley shaft JX4 may serve as a pitch rotation axis. In some embodiments, the end tool 1100 may perform a rotational motion in a vertical direction, as viewed in the drawing, around the fourth pulley shaft JX4. One or more pulleys may be disposed to be adjacent to the fourth pulley shaft JX4.

In some embodiments, a fifth pulley shaft JX5 different from the fourth pulley shaft JX4 may be disposed in the pitch hub 1108 to be spaced apart from the fourth pulley shaft JX4 and closer to the connection part 1400 than the fourth pulley shaft JX4. The fourth pulley shaft JX4 and the fifth pulley shaft JX5 may be disposed as shafts extending in parallel directions.

The end tool hub 1107 may be connected to the pitch hub 1108 and the jaws 1103. The end tool hub 1107 may be axially coupled to the pitch hub 1108 with respect to the fourth pulley shaft JX4. The end tool hub 1107 may perform a rotational motion around the fourth pulley shaft JX4 while being connected to the pitch hub 1108. That is, the end tool 1100 may perform a pitch motion as the end tool hub 1107 rotates around the fourth pulley shaft JX4 with respect to the pitch hub 1108.

In some embodiments, the jaw 1103 of the end tool 1100 may be axially coupled to the end tool hub 1107 around a first pulley shaft JX1. The first pulley shaft JX1 may serve as a yaw motion shaft of the end tool 1100. The jaw 1103 may perform a rotational motion around the first pulley shaft JX1 while being connected to the end tool hub 1107. That is, the jaw 1103 of the end tool 1100 may rotate around the first pulley shaft JX1 with respect to the end tool hub 1107, thereby performing a yaw motion.

A second pulley shaft JX2, which is adjacent to and parallel to the first pulley shaft JX1, may be disposed in the end tool hub 1107. In some embodiments, a third pulley shaft JX3 and the fourth pulley shaft JX4 may be disposed in the end tool hub 1107 in a direction different from that of the first pulley shaft JX1 and the second pulley shaft JX2, for example, in a direction intersecting or orthogonal thereto.

As a result, during a yaw motion of the end tool 1100, the jaw 1103 rotates around the first pulley shaft JX1 with respect to the end tool hub 1107. In some embodiments, during a pitch motion of the end tool 1100, the jaw 1103 coupled to the end tool hub 1107 may rotate with respect to the pitch hub 1108 as the end tool hub 1107 rotates around the fourth pulley shaft JX4 with respect to the pitch hub 1108.

In some embodiments, the third pulley shaft JX3 and the fifth pulley shaft JX5 may be pitch auxiliary pulley shafts, and the second pulley shaft JX2 may be a yaw auxiliary pulley shaft. One or more driving wires, such as a wire configured to transmit a driving force for a pitch motion or a yaw motion, may have at least one region in contact with or wound around the pulley shafts JX1, JX2, JX3, JX4, and JX5. The pulley shafts JX2, JX3, and JX5 adjacent to the fourth pulley shaft JX4, which is a pitch motion shaft, and the first pulley shaft JX1, which is a yaw motion shaft, may control paths along which the driving wires are wound around the fourth pulley shaft JX4 and the first pulley shaft JX1 to secure the efficiency of the positioning of the driving wires and stabilize the paths for transmitting forces through the driving wires.

In some embodiments, at least one region of the advancing wire 1110 may be in contact with or wound around the pulley shafts JX1, JX2, JX3, JX4, and JX5.

In some embodiments, one or more direction-changing pulley shafts, such as, a first direction-changing pulley shaft AX1 and a second direction-changing pulley shaft AX2, may be disposed in the end tool 1100. One or more direction-changing pulleys may be correspondingly disposed on each of the direction-changing pulley shafts.

For example, the first direction-changing pulley shaft AX1 and the second direction-changing pulley shaft AX2 may be disposed on the jaw 1103, specifically, on the side closer to the proximal end 1101p of the first jaw 1101, and may be positioned closer to the distal end 1101d of the first jaw 1101 than the above-described first to fifth pulley shafts JX1 to JX5.

The first direction-changing pulley shaft AX1 and the second direction-changing pulley shaft AX2 may be configured as parallel shafts, and may be disposed at different positions in a front-rear direction with respect to each other. That is, the first direction-changing pulley shaft AX1 and the second direction-changing pulley shaft AX2 may be sequentially disposed based on the distal end 1101d of the first jaw 1101 and may have partially overlapping regions.

The first direction-changing pulley shaft AX1 and the second direction-changing pulley shaft AX2 may serve as regions around which at least a portion of the advancing wires 1110 is wound or brought into contact, and may guide and align the paths of the advancing wires 1110 before the advancing wires 1110 enter any of the first to fifth pulley shafts JX1 to JX5.

As illustrated in FIG. 6, the first advancing wire 1111 and the second advancing wire 1112 may be wound and redirected in the first jaw 1101 corresponding to the first fixed pulley 1121 and the second fixed pulley 1122. The first advancing wire 1111 and the second advancing wire 1112 may be connected to the rear of the end tool 1100 via the first direction-changing pulley shaft AX1, the second direction-changing pulley shaft AX2, and at least one region of the first to fifth pulley shafts JX1 to JX5, and may further extend through the connection part 1400 to the manipulation part 1200 to allow for precise control. This allows the surgical instrument 1000 to easily implement precise motion control of the operation member 1140.

Hereinafter, the jaw 1103 of the end tool 1100 will be described in more detail.

The second jaw 1102 may be formed in a substantially bar shape and, for example, may be configured to correspond to the first jaw 1101 in at least one region.

A proximal end 1102p of the second jaw 1102 may include a region that is coupled to the first jaw 1101. For example, the second jaw 1102 may be formed to be rotatable with respect to the first jaw 1101 around a jaw coupling shaft JJX at the proximal end 1102p.

The second jaw 1102 may have various forms. For example, although not illustrated in the drawings, a plurality of anvil grooves may be formed in at least one region of the surface of the second jaw 1102 that faces the first jaw 1101. Such anvil grooves may have shapes corresponding to the shapes of the staples 1153. The anvil grooves of the second jaw 1102 may serve as supports that allow the staples 1153 to be bent when the operation member 1140 pushes the staples 1153 upward during a stapling motion.

The second jaw 1102 may include a second jaw guide groove 1102he. The second jaw guide groove 1102h may have an elongated shape extending in a longitudinal direction of the second jaw 1102.

The second jaw guide groove 1102h may be provided in the form of a groove passing through a region facing the operation member 1140, and may guide the movement of the operation member 1140. Accordingly, one region of the operation member 1140, such as at least one region of the body 1142 of the operation member 1140 or a first clamp 1146 connected thereto, may pass through the second jaw guide groove 1102h and be exposed to the outside of the second jaw 1102, and may come into contact with or apply pressure to an upper surface of the second jaw 1102.

As the operation member 1140 moves, the first clamp 1146 applies pressure on the upper surface of the second jaw 1102 and a second clamp 1147 to be described below applies pressure on a lower surface of the first jaw 1101 such that a gap between the second jaw 1102 and the first jaw 1101 decreases, allowing the second jaw 1102 to naturally remain in a closed state with respect to the first jaw 1101.

The first jaw 1101 may be formed in a substantially bar shape, and a rotation shaft may be disposed at the proximal end 1101p to allow a rotational motion. The rotation shaft may correspond to the above-described jaw coupling shaft JJX formed in the second jaw 1102. In some embodiments, the cartridge 1150 may be accommodated on a side closer to the distal end 1101d than the jaw coupling shaft JJX.

For example, the first jaw 1101 may be formed entirely in the shape of a hollow box with one surface (an upper surface) removed, and a cartridge accommodation part 1101a configured to accommodate the cartridge 1150 may be formed inside. That is, the first jaw 1101 may be formed in an approximately “U” shape in cross section.

A first jaw guide groove 1101h may be formed in a bottom surface of the first jaw 1101, the bottom surface being opposite to an upper open region formed by removing one surface. For example, the first jaw guide groove 1101h may be guide a linear motion of the operation member 1140.

The first jaw guide groove 1101h may be provided in the form of a groove passing through a region facing the operation member 1140, and may guide the movement of the operation member 1140. Accordingly, one region of the operation member 1140, such as at least one region of the body 1142 of the operation member 1140, or the second clamp 1147 connected thereto, may pass through the first jaw guide groove 1101h and be ejected to the outside of the first jaw 1101.

When the operation member 1140 moves forward, the second clamp 1147 may pass through the first jaw guide groove 1101h to be exposed to the outside of the first jaw 1101, and may come into contact with the lower surface of the first jaw 1101 or apply pressure thereto. As the operation member 1140 moves, the second clamp 1147 applies pressure on the lower surface of the first jaw 1101 and the first clamp 1146 applies pressure on the upper surface of the second jaw 1102 such that the gap between the second jaw 1102 and the first jaw 1101 decreases, allowing the second jaw 1102 to naturally remain in a closed state with respect to the first jaw 1101.

In an optional embodiment, the first jaw 1101 may include a window 1101b. After operating the operation member 1140 or using the end tool 1100, the second clamp 1147 of the operation member 1140 may be positioned at a location corresponding to the window 1101b, and the coupled state of the first jaw 1101 and the operation member 1140 may be released.

The first jaw 1101 may include a front space 1101c located ahead of the cartridge accommodation part 1101a.

For example, the front space 1101c may be positioned closer to the distal end 1101d of the first jaw 1101 than the cartridge accommodation part 1101a. Fixed pulleys 1120, such as, the first fixed pulley 1121 and the second fixed pulley 1122, may be disposed in the front space 1101c.

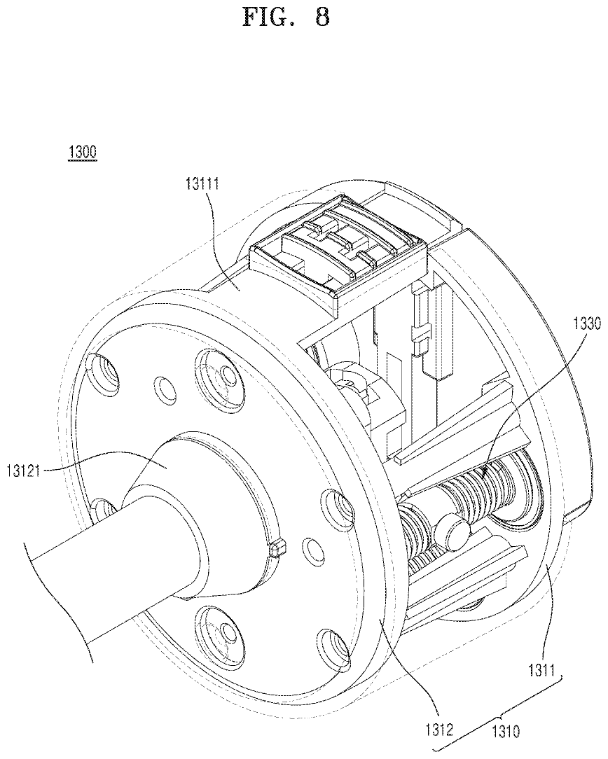

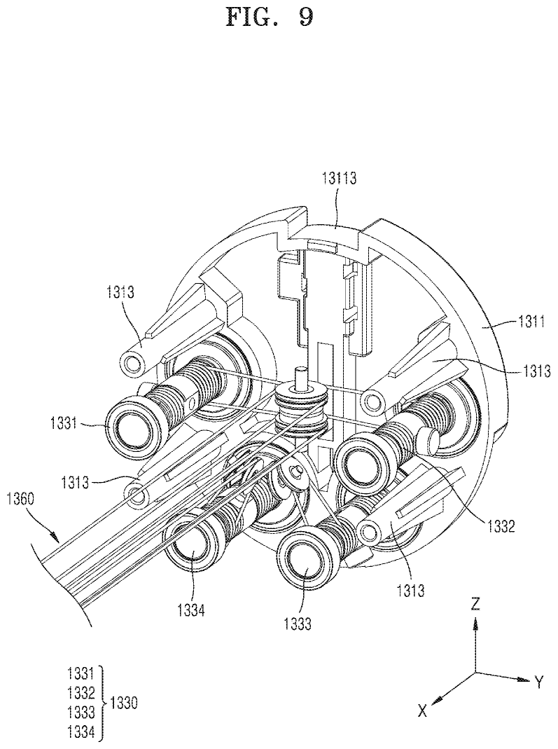

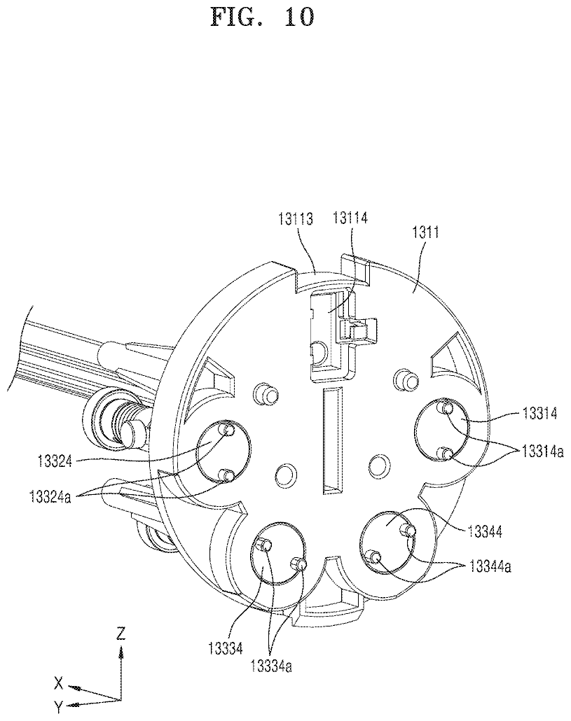

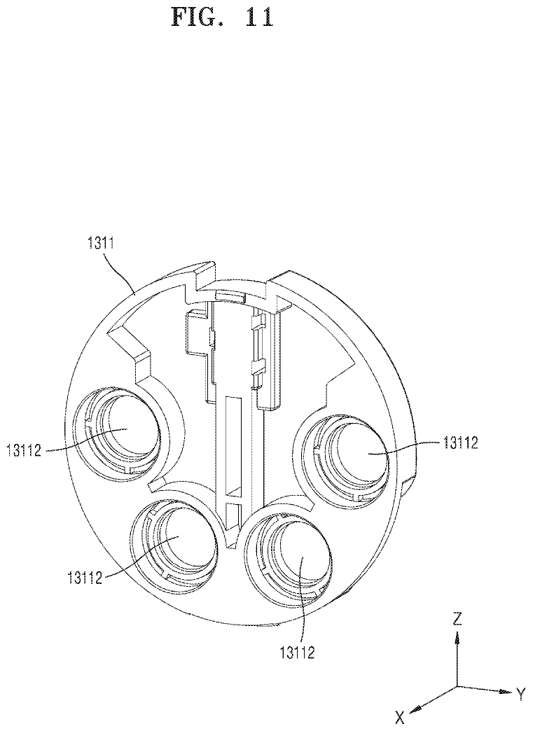

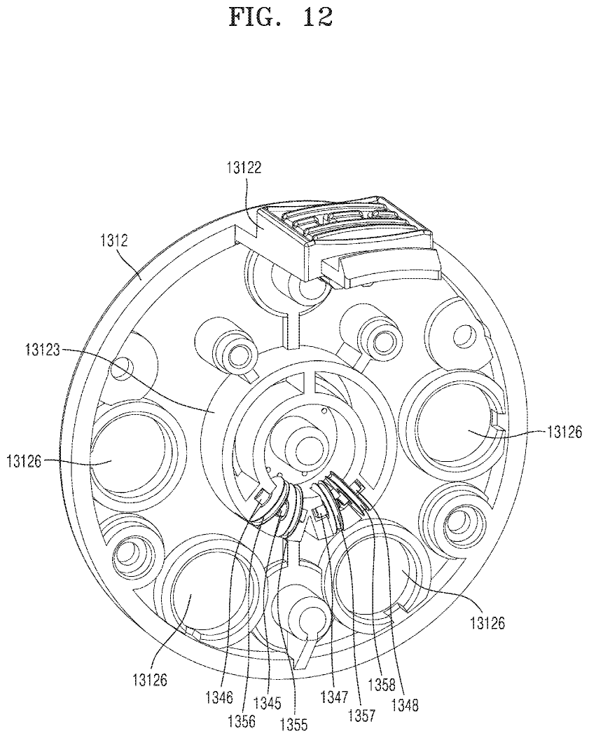

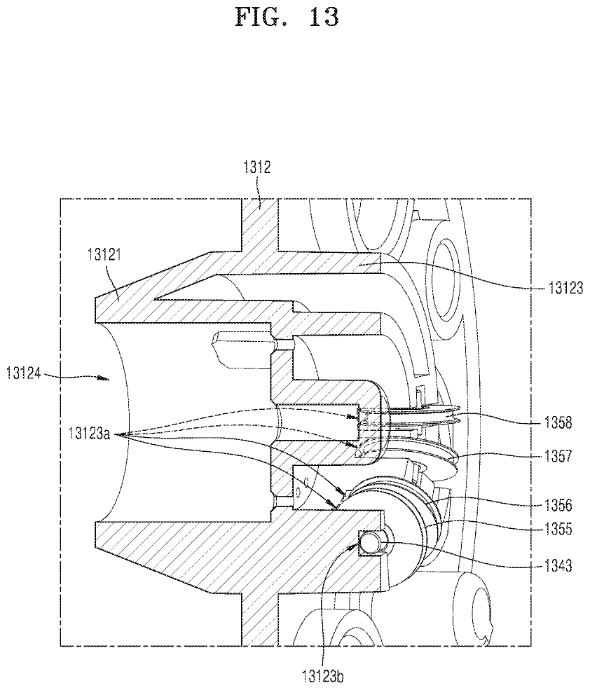

FIG. 8 is a perspective view illustrating the power transmission part 1300 of FIG. 2. FIG. 9 is a view illustrating an internal structure of the power transmission part 1300 of FIG. 8, and FIG. 10 is a rear view of the power transmission part 1300 of FIG. 8. FIG. 11 is a perspective view illustrating a first pulley frame 1311 of the power transmission part 1300 of FIG. 8, FIG. 12 is a perspective view illustrating a second pulley frame 1312 of the power transmission part 1300 of FIG. 8, and FIG. 13 is a perspective cross-sectional view illustrating a side cross-section of the second pulley frame 1312 of FIG. 12.

Referring to FIGS. 8 to 13, the power transmission part 1300 may include a pulley frame 1310, a drive pulley unit, an auxiliary pulley unit, and a wire unit.

The drive pulley unit may refer to either the drive pulley itself or a plurality of drive pulleys. That is, the drive pulley unit may include a plurality of drive pulleys, and in describing features common to each drive pulley, a representative drive pulley will be referred to as a drive pulley 1330 for convenience of description.

Similarly, the wire unit may include a plurality of wires, and in describing features common to each wire, a representative wire will be referred to as a wire 1360 for convenience of description.

In addition, the auxiliary pulley unit may include a plurality of auxiliary pulleys, and in describing features common to each auxiliary pulley, a representative auxiliary pulley will be referred to as an auxiliary pulley 1350 for convenience of description.

The pulley frame 1310 may be connected to the power generation part 1500. For example, the pulley frame 1310 may be coupled to the housing of the manipulation part 1200 and may be connected to the power generation part 1500 provided in the manipulation part 1200. For example, the pulley frame 1310 may be connected to a pulley coupling plate of the power generation part 1500 to be described later. Accordingly, the pulley frame 1310 may support the drive pulley 1330 so that the drive pulley 1330 to be described later may be stably connected to a drive motor of the power generation part 1500.

In some embodiments, the pulley frame 1310 may accommodate the drive pulley 1330 to allow for axial rotation. Accordingly, the drive pulley 1330 is connected to the drive motor and may rotate by receiving driving force generated from the drive motor.

The pulley frame 1310 may include the first pulley frame 1311 and the second pulley frame 1312. The first pulley frame 1311 and the second pulley frame 1312 may be members that are connected to each other.

The first pulley frame 1311 may be a portion coupled to the power generation part 1500, and the second pulley frame 1312 may be a portion coupled to the shaft 1400.

In other words, the pulley frame 1310 may be interposed between the shaft 1400 and the power generation part 1500, the first pulley frame 1311 may be positioned at the proximal end, and the second pulley frame 1312 may be positioned at the distal end.

The first pulley frame 1311 may include first drive pulley coupling parts 13112 each accommodating at least a portion of the drive pulley 1330. The first drive pulley coupling parts 13112 may be formed at positions corresponding to drive motors (not shown) of the power generation part 1500. Each of the first drive pulley coupling parts 13112 may be formed in a hollow shape passing through the first pulley frame 1311. For example, a hollow-inner surface of the first drive pulley coupling part 13112 may be formed to receive bearings 13303 of the drive pulley 1330.

A plurality of first drive pulley coupling parts 13112 may be provided to correspond to the number of drive pulleys 1330. The plurality of first drive pulley coupling parts 13112 may be symmetrically disposed with respect to the center of the first pulley frame 1311.

The first pulley frame 1311 may include one or more fastening member receiving grooves 13113 along its side perimeter. Each of the fastening member receiving grooves 13113 may be configured to accommodate a fastening member 13122 that is formed by extending from the second pulley frame 1312. For example, the fastening member receiving groove 13113 may receive an end portion of the fastening member 13122. For example, the fastening member receiving groove 13113 may be formed wider than a width of the fastening member 13122, and may have a certain depth to allow the end portion of the fastening member 13122 to move in a thickness direction.

In some embodiments, the first pulley frame 1311 may further include a data transmission and reception part 13114 on a rear surface thereof, which is opposite to the surface facing the second pulley frame 1312, the data transmission and reception part 13114 being electrically connected to the manipulation part 1200 or another device.

The data transmission and reception part 13114 may further include a memory. The memory may store programs and instructions for processing and control by a processor, and may also store data input from or output to a device.

For example, the memory may include at least one type of storage medium, such as a flash memory type, a multimedia card micro type memory, a card-type memory (e.g., a secure digital (SD) or extreme digital (XD) memory), a random access memory (RAM), a static random access memory (SRAM), a read-only memory (ROM), an electrically erasable programmable read-only memory (EEPROM), and a programmable read-only memory (PROM).

The data transmission and reception part 13114 can read and write data, and may store information related to the end tool 1100 while providing information to prevent reuse. For example, the data transmission and reception part 13114 may store information including a total length of a reload, which includes the end tool 1100, the shaft 1400, and the power transmission part 1300, the specifications of the reload including the type of the end tool 1100, the number of uses of the reload, and the like.

The second pulley frame 1312 may include a shaft coupling part 13121, an extension portion 13123 of the shaft coupling part, the fastening member 13122, and a second drive pulley coupling part 13126.

The shaft coupling part 13121 may be formed to protrude from the second pulley frame 1312 toward the distal end. Although not shown in the drawing, the shaft coupling part 13121 may have a shaft insertion hole that receives at least a portion of the shaft 1400. An inner diameter of the shaft coupling part 13121 may be configured to correspond to an outer diameter of the shaft 1400, allowing the shaft 1400 to be inserted, and the end portion of the shaft 1400 may be inserted into the shaft coupling part 13121.

The second pulley frame 1312 may have the extension portion 13123 of the shaft coupling part 13121 formed on a surface thereof opposite to the surface on which the shaft coupling part 13121 is formed. That is, the extension portion 13123 may be formed to protrude toward the proximal end. In addition, a hollow may be formed in the extension portion 13123. “In other words, the shaft coupling part 13121 may be formed in a shape that passes through the second pulley frame 1312 and is exposed on both front and rear surfaces of the second pulley frame 1312.

As shown in FIG. 13, the extension portion 13123 of the shaft coupling part 13121 may include auxiliary pulley receiving grooves 13123a and rotation shaft fixing grooves 13123b. The auxiliary pulley receiving grooves 13123a may respectively receive at least portions of the auxiliary pulleys 1355, 1356, 1357, and 1358. In addition, the rotation shaft fixing grooves 13123b may receive auxiliary pulley rotation shafts 1345, 1346, 1347, and 1348.

For example, each of the auxiliary pulley receiving grooves 13123a may be formed in the shape of a slit corresponding to a thickness of a respective one of the auxiliary pulleys 1355, 1356, 1357, and 1358.

The rotation shaft fixing grooves 13123b may be formed in a structure open in a lateral direction, such that the auxiliary pulley rotation shafts 1345, 1346, 1347, and 1348 are fixed in a rotational axis direction but movable in the lateral direction.

The auxiliary pulley receiving groove 13123a and the rotation shaft fixing groove 13123b may be formed along a perimeter of a shaft insertion hole 13124. The auxiliary pulley rotation shafts 1345, 1346, 1347, and 1348 are disposed to be perpendicular to a central axis of the shaft 1400, but may not be parallel to each other.

In some embodiments, the fastening member 13122 of the second pulley frame 1312 may be formed to extend from the second pulley frame 1312 as described above. For example, the fastening member 13122 may be formed in a snap-fit structure. In some embodiments, an end portion of the fastening member 13122 may be formed in the shape of a hook so as to be coupled to a fastening groove formed in the manipulation part 1200 or another device. The fastening member 13122 may also be formed to be at least partially receivable in the fastening member receiving groove 13113 of the first pulley frame 1311.

A frame coupling part 1313 may be positioned between the first pulley frame 1311 and the second pulley frame 1312. The first pulley frame 1311 may be fixedly coupled to the second pulley frame 1312 by the frame coupling part 1313. The frame coupling part 1313 may be formed to extend from one of the first pulley frame 1311 and the second pulley frame 1312 toward the other. “For example, the frame coupling part 1313 may have a generally columnar shape, and a plurality of frame coupling parts 1313 may be disposed at intervals in a circumferential direction of the pulley frame 1310. Accordingly, the plurality of frame coupling parts 1313 connect and couple the first pulley frame 1311 to the second pulley frame 1312, and may support a side surface of the pulley frame 1310.

The second drive pulley coupling part 13126 provided in the second pulley frame 1312 may be positioned to correspond to the first drive pulley coupling part 13112 formed in the first pulley frame 1311. Accordingly, one end portion of the drive pulley 1330 or a rotation axis of the drive pulley 1330 may be coupled to the first pulley frame 1311, and another end portion thereof may be coupled to the second pulley frame 1312.

For example, the bearings 13303 may be coupled to both end portions of the drive pulley 1330, and coupled to the first drive pulley coupling part 13112 and the second drive pulley coupling part 13126. Alternatively, the bearings 13303 may be coupled to both end portions of the rotation axis of the drive pulley 1330, and coupled to the first drive pulley coupling part 13112 and the second drive pulley coupling part 13126.

In other words, it may be said that the drive pulley 1330 is positioned between the first pulley frame 1311 and the second pulley frame 1312, and the first pulley frame 1311 and the second pulley frame 1312 are positioned to be spaced apart by a length of the drive pulley 1330.

However, the shape and structure of the pulley frame 1310 are not necessarily limited thereto, and the pulley frame 1310 may have various structures for positioning the drive pulley 1330 parallel to the shaft 1400.

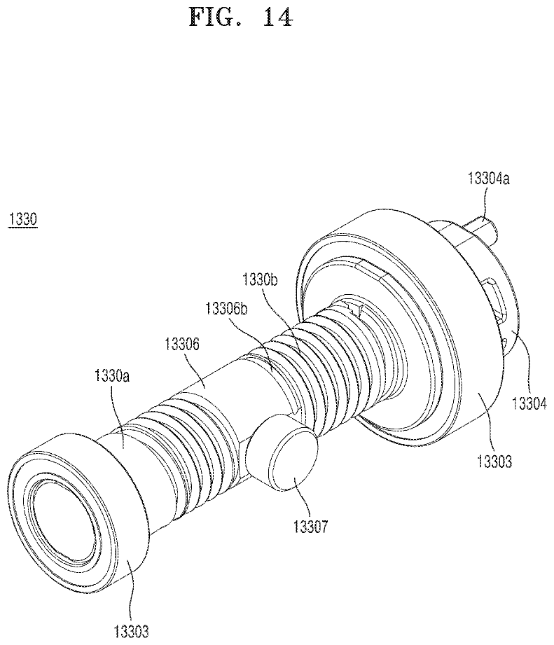

FIG. 14 is a perspective view illustrating the drive pulley 1330 of the power transmission part 1300 of FIG. 8.

Referring again to FIGS. 8 to 10, the drive pulley 1330 may be accommodated in the pulley frame 1310 and may rotate around its axis by receiving power generated from the power generation part 1500. The rotation axis of the drive pulley 1330 may be positioned parallel to the shaft 1400. When a plurality of drive pulleys 1330 are provided, rotation axes of the plurality of drive pulleys 1330 may be positioned parallel to each other.

The power transmission part 1300 may include firing drive pulleys around which an advancing wire and a retraction wire are wound, specifically, a first firing drive pulley 1331 and a second firing drive pulley 1332. In addition, the power transmission part 1300 may include a yaw drive pulley 1333 around which a yaw wire is wound, and a pitch drive pulley 1334 around which a pitch wire is wound.

The first firing drive pulley 1331 and the second firing drive pulley 1332 are drive pulleys associated with the movement of the operation member 1140 of the end tool 1100, and the advancing wire and the retraction wire are wires associated with the movement of the operation member 1140 of the end tool 1100.

The yaw drive pulley 1333 is a drive pulley associated with a yaw rotation of the end tool 1100, and the yaw wire is a wire associated with the yaw rotation of the end tool 1100.

The pitch drive pulley 1334 is a drive pulley associated with a pitch rotation of the end tool 1100, and the pitch wire is a wire associated with the pitch rotation of the end tool 1100.

As described above, in describing features common to the first firing drive pulley 1331, the second firing drive pulley 1332, the yaw drive pulley 1333, and the pitch drive pulley 1334, a representative pulley will be referred to as the drive pulley 1330 for convenience of description, and each of the drive pulleys will be described in detail below.

The wire 1360 is connected to a side surface of the drive pulley 1330, and may be wound or unwound in response to the rotation of the drive pulley 1330. That is, one end portion of the wire 1360 is connected to the drive pulley 1330 and another end portion thereof is connected to the end tool 1100, thereby allowing power generated by the power generation part 1500 to be transmitted to the end tool 1100.

Referring to FIG. 14, the drive pulley 1330 of the power transmission part 1300 may include a body 1330a, a wire fixing part 13306, a pulley plate 13304, and the bearings 13303.

The body 1330a is formed in a cylindrical shape and may form the center of rotation of the drive pulley 1330. The pulley plate 13304 may be coupled to one end portion of the body 1330a. The drive pulley 1330 may receive a rotational force applied to the pulley plate 13304 and rotate around its axis.

A groove 1330b may be formed in a side surface of the body 1330a. For example, the groove 1330b with a threaded shape may be formed on an outer circumferential surface of the drive pulley 1330, and the groove 1330b may be shaped to allow the wire 1360 to wind around the body 1330a of the drive pulley 1330.

The bearings 13303 may be positioned at one end portion or both end portions of the drive pulley 1330. The bearings 13303 support the axis of the drive pulley, allowing the drive pulley 1330 to rotate smoothly around its axis within the pulley frame 1310.

For example, the bearings 13303 are provided as a pair and may be inserted into the first drive pulley coupling part 13112 provided in the first pulley frame 1311 and the second drive pulley coupling part 13126 provided in the second pulley frame 1312, respectively. Accordingly, the drive pulley 1330 may rotate around its axis within the pulley frame 1310.

Although not specifically shown in the drawing, the wire 1360 may be fixed to the drive pulley 1330 in various ways. For example, the wire 1360 may be fixed to the drive pulley 1330 by a fixing member, a bolt, or the like. Here, the fixing member may be formed in various shapes, such as a ball shape, a tube shape, or the like as necessary.

For example, one end portion of the wire 1360 is coupled to a fixing member (not shown), and the fixing member may fix the one end portion of the wire 1360 to the drive pulley 1330 by being engaged across the drive pulley 1330.

Alternatively, one end portion of the wire 1360 may be fastened to the drive pulley 1330 using a bolt or the like. For example, one end portion of the wire 1360 may be wound beneath a bolt head, and the bolt may be fastened to the drive pulley 1330, thereby allowing the wire 1360 to be fixed to the drive pulley 1330. In an optional embodiment, a washer may be placed beneath the bolt, which can press the wire 1360 more stably.

In some embodiments, a pair of wires may be wound around the drive pulley 1330, with one wire being fastened using a bolt and another wire being fastened with a fastening member.

For example, one end portion of the one wire may be wound around the drive pulley 1330 a certain number of times, and then the fixing member may be fastened to the drive pulley 1330 to fix the wire to the drive pulley 1330. In this state, when the drive pulley 1330 rotates, the one wire may wind further around the drive pulley 1330. Accordingly, the one wire may be wound around the drive pulley 1330 under an initial tension.

In this state, the another wire may be wound around the drive pulley 1330 and fixed to the drive pulley 1330 by a bolt. The another wire may be pulled before being wound around the bolt and fixed to the drive pulley 1330 by the bolt, while under the initial tension.

In addition, since the initial tension is applied to the wire, the wire may rotate the bolt in a specific direction. At this point, a direction in which the wire is wound is opposite to a direction in which the bolt is fastened, and as the wire is pulled, the bolt may be tightened more.

In some embodiments, when both wires are fixed to the drive pulley 1330 with a fixing member, it may be difficult to apply initial tension to the wire unless the fixing member is fixed in a precise position. As described above, by fixing one wire with a fixing member and another wire with a bolt, the initial tension may be smoothly applied to the wires.

As described above, the surgical instrument 1000 according to an embodiment of the present disclosure may allow for easier process control in fixing different wires to the drive pulley 1330, thereby improving productivity.

The wire fixing part 13306 may be formed on one side of the body 1330a. In some embodiments, the drive pulley 1330 may further include a fixing pin 13307 inserted into the wire fixing part 13306. Here, the fixing pin 13307 may serve the role of the above-described bolt, and the wire fixing part 13306 may additionally serve to fix the wire.

In some embodiments, one or more grooves 13306b may be provided on one side of the wire fixing part 13306. A portion of the wire may be positioned in the groove 13306b of the wire fixing part 13306. Accordingly, one end portion of the wire wound around the drive pulley 1330 may be fixed to the drive pulley 1330 by being compressed between the wire fixing part 13306 and the body 1330a.

The pulley plate 13304 may be coupled to one end portion of the body 1330a. The pulley plate 13304 may further include a protrusion 13304a protruding outward. The protrusion 13304a may be coupled to a motor plate provided on the drive motor to be described later.

The protrusion 13304a ensures stable fastening between the pulley plate 13304 and the motor plate, thereby allowing a rotational force of the drive motor to be transmitted to the drive pulley 1330 through the pulley plate 13304. However, the concept of the present disclosure is not limited thereto, and it is, of course, possible for a groove to be formed in the pulley plate 13304 and a protrusion to be formed in the motor plate, which enables the groove in the pulley plate 13304 and the protrusion in the motor plate to be fastened together.

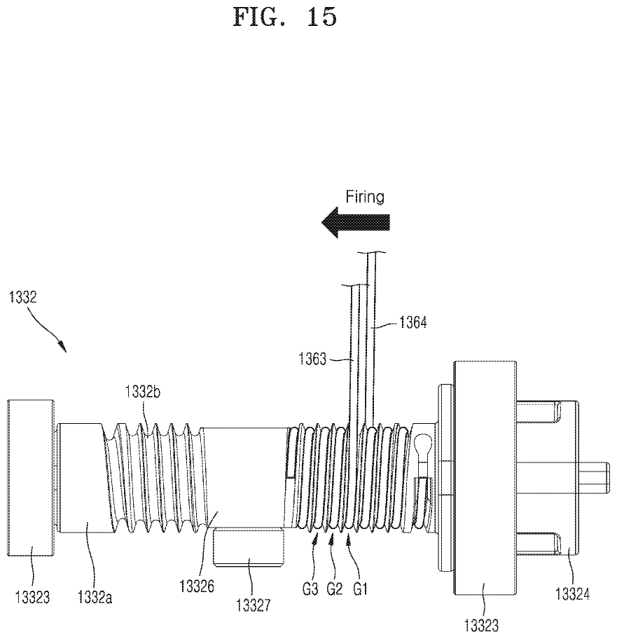

FIGS. 15 to 17 are views illutrating a process in which a pair of wires 1363 and 1364 are wound around and unwound from the second firing drive pulley 1332 of FIG. 9.

Referring to FIG. 9 and FIGS. 15 to 17 together, the wire may be wound around an outer circumferential surface of the drive pulley 1330 or unwound from the outer circumferential surface of the drive pulley 1330 in response to the rotation of the drive pulley 1330.

The wire unit may include a pair of wires distinguished as a first wire and a second wire. The first wire may be wound around the drive pulley 1330 in a first direction, and the second wire may be wound around the drive pulley 1330 in a second direction opposite to the first direction. In some embodiments, in response to the rotation of the drive pulley 1330 in either direction, one of the first wire and the second wire may be wound around the drive pulley 1330, and another one thereof may be unwound from the drive pulley 1330.

For example, when the first wire is wound around the drive pulley 1330 in a clockwise direction and the second wire is wound around the drive pulley 1330 in a counterclockwise direction, when the drive pulley 1330 rotates in the clockwise direction, the first wire may be further wound around the drive pulley 1330, and the second wire may be unwound from the drive pulley 1330. In some embodiments, when the drive pulley 1330 rotates in the counterclockwise direction, the first wire may be unwound from the drive pulley 1330, and the second wire may be further wound around the drive pulley 1330.

As such, during the rotation of the drive pulley 1330 in one direction, the first wire and the second wire may move in different directions. That is, due to the rotation of the drive pulley 1330 in one direction, one wire may be pulled and another wire may be unwound.

For example, the advancing wire and the retraction wire connected to the first firing drive pulley 1331 and the second firing drive pulley 1332 are connected to the operation member 1140 of the end tool 1100, and each of the advancing wire and the retraction wire connected to the same operation member 1140 may move in opposite directions at a 1:1 ratio.

For convenience of description, the following description will focus on the second firing drive pulley 1332, although the same principle may also apply to the first firing drive pulley 1331, the yaw drive pulley 1333, and the pitch drive pulley 1334.

As the second firing drive pulley 1332 rotates in one direction, the advancing wire may be wound around the second firing drive pulley 1332, causing the operation member of the end tool 1100 to move forward. In some embodiments, as the second firing drive pulley 1332 rotates in a direction opposite to the one direction, the retraction wire may be wound around the second firing drive pulley 1332, causing the operation member 1140 of the end tool 1100 to move backward.

In some embodiments, when a pair of wires are wound around the drive pulley 1330 in opposite directions, the position at which the wire is wound may move in a lateral direction along the drive pulley 1330 as the wire is wound or unwound.

FIG. 15 is a view illustrating a state in which a pair of wires are wound around the second firing drive pulley 1332. Here, the pair of wires may include a second retraction wire 1363 and a second advancing wire 1364.

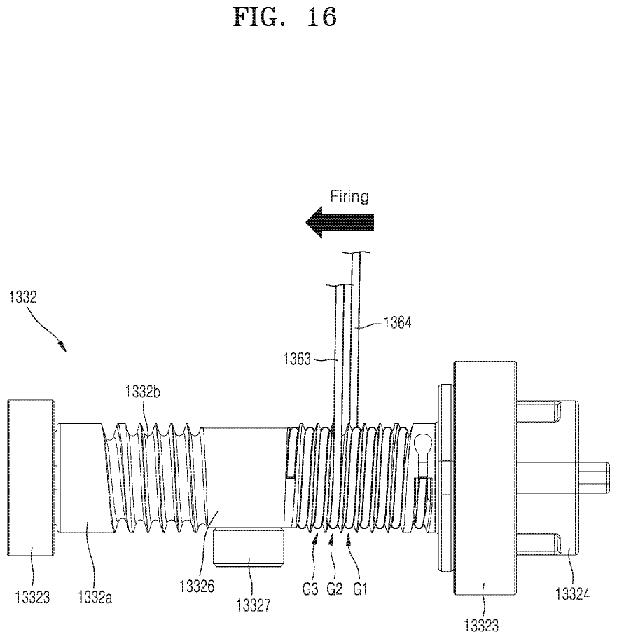

FIG. 16 is a view illustrating a state in which the second retraction wire 1363 is unwound from the second firing drive pulley 1332, and the second advancing wire 1364 is further wound around the second firing drive pulley 1332 as the second firing drive pulley 1332 rotates in one direction.

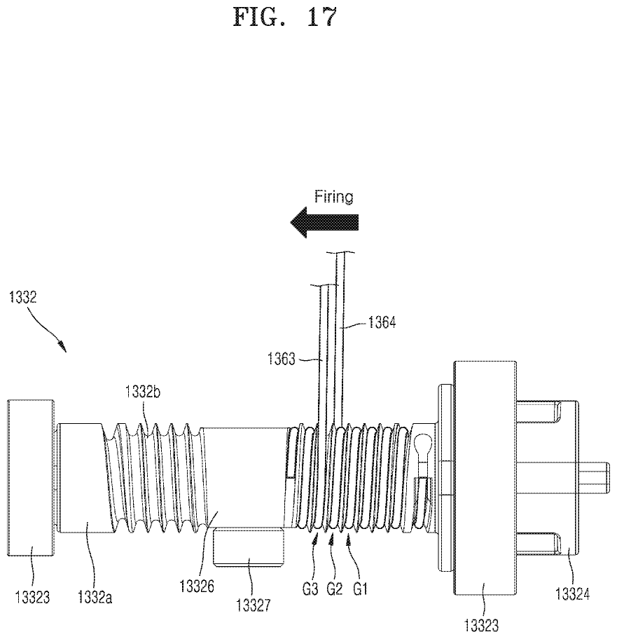

FIG. 17 is a view illustrating a state in which the second retraction wire 1363 is further unwound from the second firing drive pulley 1332, and the second advancing wire 1364 is further wound around the second firing drive pulley 1332 as the second firing drive pulley 1332 rotates in one direction.

During the process from FIG. 15 to FIG. 17, the second firing drive pulley 1332 may be in a state of having completed one full rotation. That is, for each full rotation of the second firing drive pulley 1332, the second retraction wire 1363 may be unwound one full rotation from the second firing drive pulley 1332, and the second advancing wire 1364 may be wound one full rotation around the second firing drive pulley 1332.

In other words, the drive pulley 1330 forms a continuous groove 1330b around which the first and second wires are wound, and as the drive pulley 1330 rotates, one wire may be wound into a position from which another wire is unwound.

For example, as shown in FIG. 15, the second retraction wire 1363 and the second advancing wire 1364 are wound around a first groove G1, and as the second firing drive pulley 1332 rotates in one direction, the second retraction wire 1363 and the second advancing wire 1364 are wound into a second groove G2. As the second firing drive pulley 1332 continues to rotate, the second retraction wire 1363 and the second advancing wire 1364 are wound into a third groove G3.

In other words, in the drive pulley 1330 according to an embodiment of the present disclosure, not just one wire is wound in the groove 1330b, but both the first wire and the second wire may be wound together. In other words, the pair of wires connected to one drive pulley 1330 may share the groove 1330b.

Hereinafter, the arrangement structure of the drive pulley 1330, the auxiliary pulley 1350, and the wire 1360 of the power transmission part 1300 according to an embodiment of the present disclosure will be described in detail.

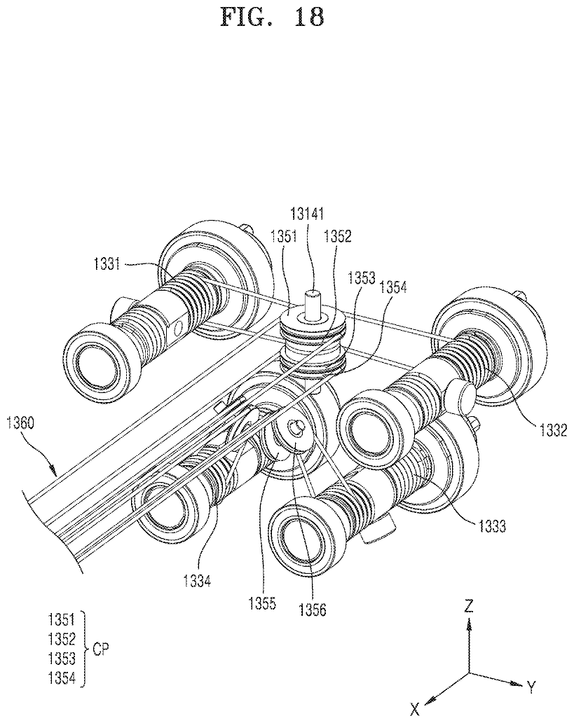

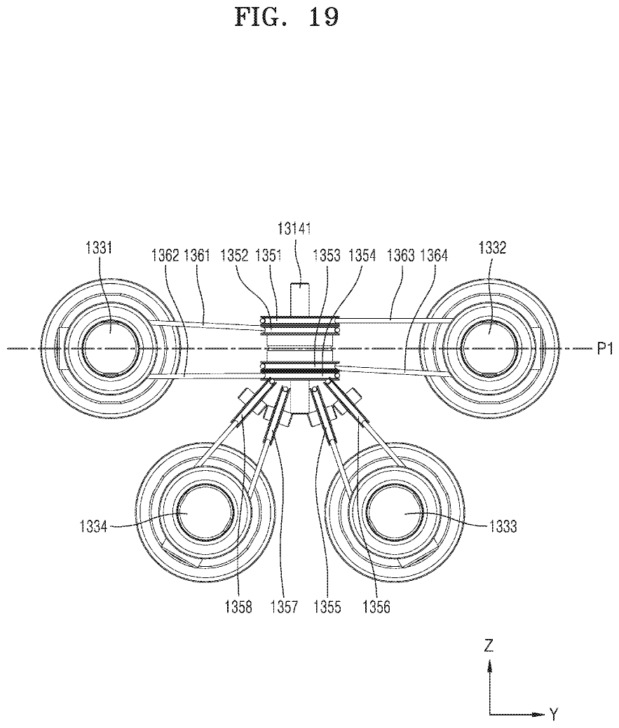

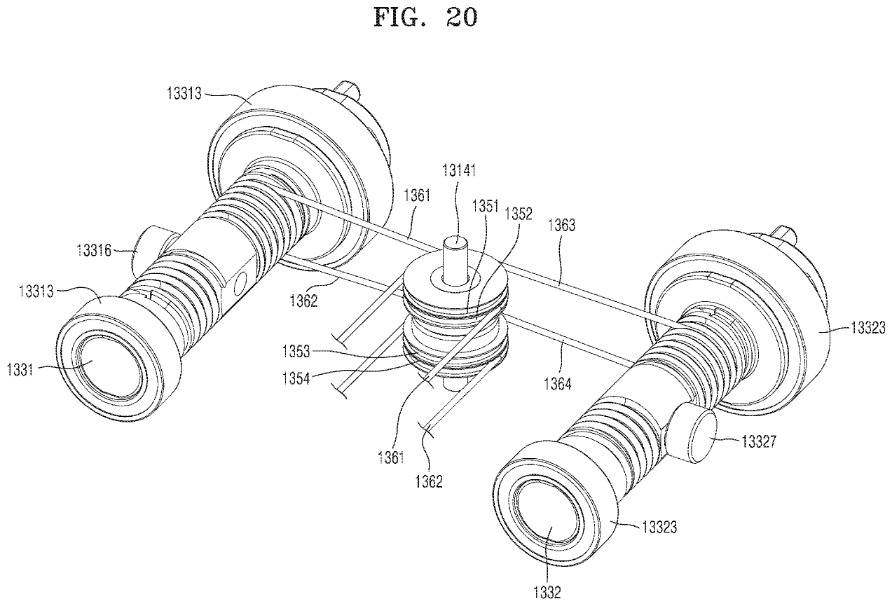

FIG. 18 is a perspective view illustrating the power transmission part 1300 of FIG. 8 with the first pulley frame 1311 removed. FIG. 19 is a front view of the power transmission part 1300 of FIG. 18, and FIG. 20 is a perspective view illustrating the wires connected to the drive pulley 1330 and a center auxiliary pulley CP in the power transmission part 1300 of FIG. 18.

Referring to FIGS. 18 to 20, the drive pulley unit may include the first firing drive pulley 1331, the second firing drive pulley 1332, the yaw drive pulley 1333, and the pitch drive pulley 1334.

The first firing drive pulley 1331 may be a drive pulley around which a first advancing wire 1361 and a first retraction wire 1362 are wound. The second firing drive pulley 1332 may be a drive pulley around which the second advancing wire 1364 and the second retraction wire 1363 are wound. The yaw drive pulley 1333 may be a drive pulley around which yaw wires 1365 and 1366 are wound. In addition, the pitch drive pulley 1334 may be a drive pulley around which pitch wires 1367 and 1368 are wound.

The first firing drive pulley 1331 and the second firing drive pulley 1332 may be symmetrically disposed with respect to the central axis of the shaft 1400. In addition, the yaw drive pulley 1333 and the pitch drive pulley 1334 may be spaced apart from the first firing drive pulley 1331 and the second firing drive pulley 1332, and may be positioned parallel to the shaft 1400. In the drawing, the yaw drive pulley 1333 and the pitch drive pulley 1334 are illustrated as being positioned below the first firing drive pulley 1331 and the second firing drive pulley 1332, but the present disclosure is not limited thereto, and the yaw drive pulley 1333 and the pitch drive pulley 1334 may be positioned within the pulley frame 1310 to be parallel to the first firing drive pulley 1331 and the second firing drive pulley 1332.

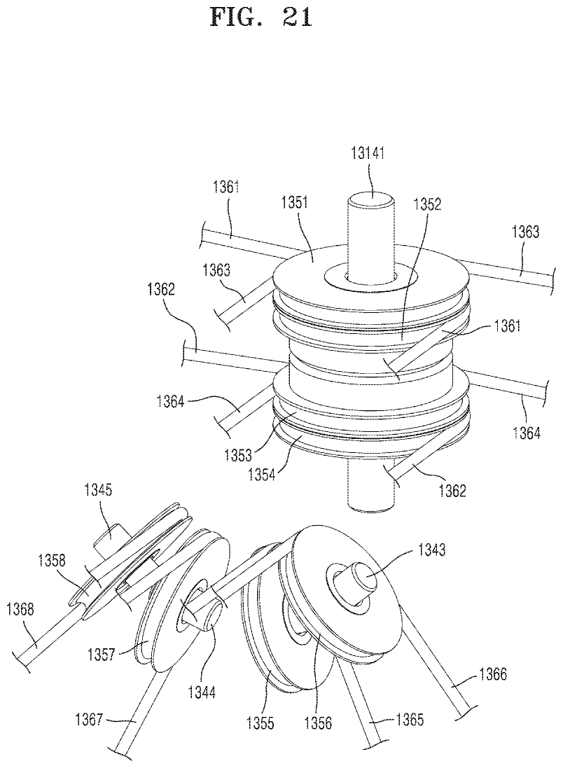

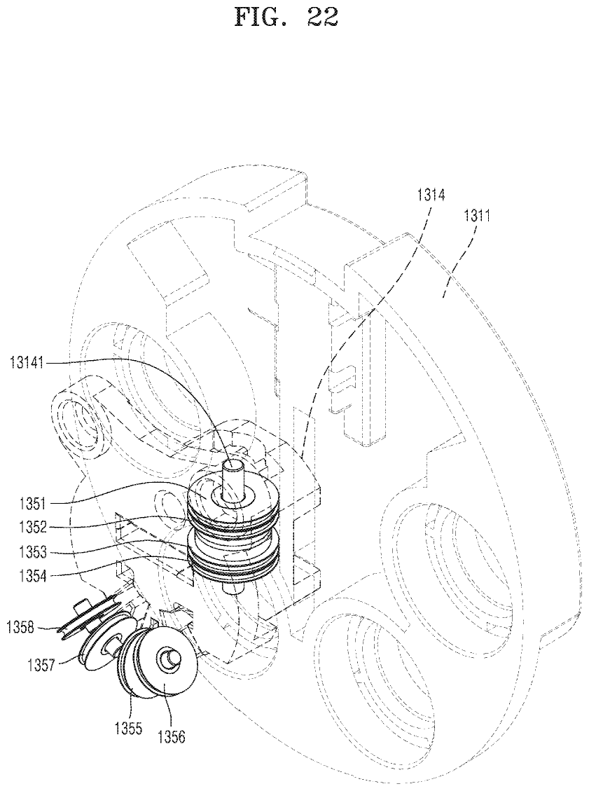

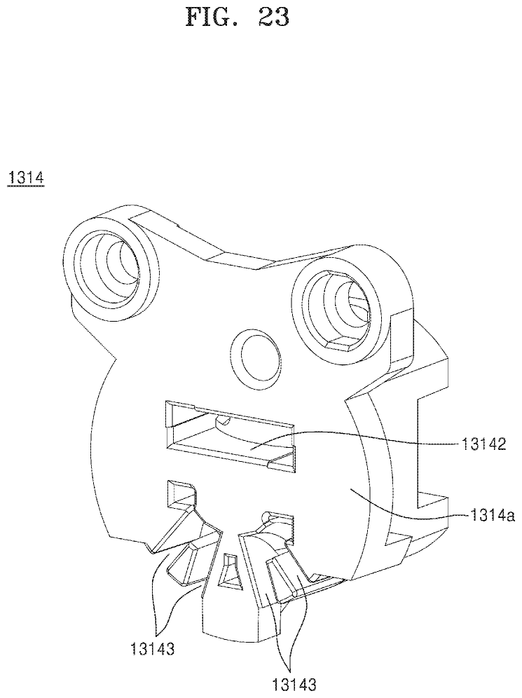

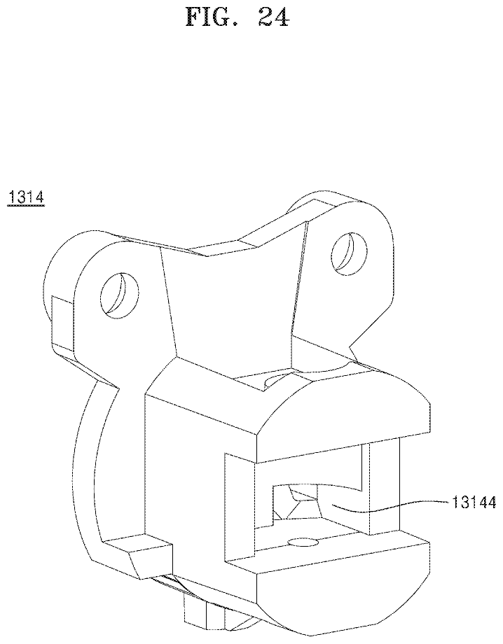

FIG. 21 is a perspective view illustrating a state in which the wire 1360 is wound around the auxiliary pulley 1350 in the power transmission part 1300 of FIG. 8, and FIG. 22 is a view illustrating the auxiliary pulley 1350 positioned in the pulley frame 1310 of the power transmission part 1300 of FIG. 8. FIGS. 23 and 24 are perspective views illustrating an auxiliary pulley support member 1314 of FIG. 22.

Referring to FIGS. 18 to 24, the auxiliary pulley 1350 may be disposed so as to be partially in contact with the wire 1360, thereby redirecting the path of the wire 1360 extending from the drive pulley 1330 and guiding the wire into the shaft 1400.

In other words, the wire 1360 wound around the drive pulley 1330 may pass through the auxiliary pulley 1350 and be positioned inside the shaft 1400. The wire 1360 may pass through the inside of the shaft 1400 and be connected to the pulleys of the end tool 1100.

The auxiliary pulley unit may include a pair of auxiliary pulleys. The pair of auxiliary pulleys may be disposed spaced apart from each other so as to correspond to a diameter of the drive pulley 1330 around which a pair of wires are wound. The pair of wires may be wound around one drive pulley 1330 and extend therefrom, and may be positioned to be substantially parallel to each other. In this case, each wire extending from the drive pulley 1330 passes through the respective auxiliary pulley 1350, which are spaced apart from each other, so that the wires are positioned sufficiently apart from each other.

The auxiliary pulley unit may include the center auxiliary pulley CP disposed adjacent to the central axis of the shaft 1400. The center auxiliary pulley CP may be disposed adjacent to an extension line of the shaft 1400. In other words, the center auxiliary pulley CP may be positioned between the first firing drive pulley 1331 and the second firing drive pulley 1332.

The center auxiliary pulley CP may be disposed in the auxiliary pulley support member 1314. The center auxiliary pulley CP may be received in the center auxiliary pulley fixing part 13144 of the auxiliary pulley support member 1314. Here, the center auxiliary pulley CP is axially coupled to a center auxiliary pulley rotation shaft 13141, and the center auxiliary pulley rotation shaft 13141 may be coupled to the auxiliary pulley support member 1314.

The center auxiliary pulley rotation shaft 13141 may be perpendicular to the central axis of the shaft 1400. In some embodiments, the center auxiliary pulley rotation shaft 13141 may be perpendicular to an imaginary plane P1 that includes rotation axes of a pair of drive pulleys symmetrically disposed with respect to the central axis of the shaft 1400.

That is, since the center auxiliary pulley CP rotates around the center auxiliary pulley rotation shaft 13141, a rotational plane on which the center auxiliary pulley CP rotates may be parallel to the imaginary plane P1.

Accordingly, the wires extending from the first firing drive pulley 1331 and the second firing drive pulley 1332 may pass through the center auxiliary pulley CP, thereby changing direction by approximately 90° and entering the inside of the shaft 1400. At this point, the wires may extend through a wire guide part 13142 of the auxiliary pulley support member 1314.

In summary, according to an embodiment of the present disclosure, the wire 1360 extending from the inside of the shaft 1400 to the power transmission part 1300 by the auxiliary pulley 1350 of the power transmission part 1300 may be stably wound around the drive pulley 1330, which is disposed parallel to the shaft 1400.

The center auxiliary pulley CP may include a first auxiliary pulley 1351, a second auxiliary pulley 1352, a third auxiliary pulley 1353, and a fourth auxiliary pulley 1354, all of which are axially coupled around the same rotation shaft.

The first auxiliary pulley 1351 and the second auxiliary pulley 1352 may be disposed on one side of the central axis of the shaft 1400, and the third auxiliary pulley 1353 and the fourth auxiliary pulley 1354 may be disposed on another side of the central axis of the shaft 1400.

In other words, the first auxiliary pulley 1351 and the second auxiliary pulley 1352 may be disposed on one side of an imaginary plane P1 that includes a rotation axis of each of the pair of drive pulleys, and the third auxiliary pulley 1353 and the fourth auxiliary pulley 1354 may be disposed on another side of the imaginary plane P1.

As described above, in the power transmission part 1300 according to an embodiment of the present disclosure, a pair of wires may be wound in opposite directions around one drive pulley 1330.

For example, the drive pulley unit may include the first firing drive pulley 1331 and the second firing drive pulley 1332 that are symmetrically disposed with respect to the central axis of the shaft 1400. The first advancing wire 1361 and the first retraction wire 1362 may be wound around the first firing drive pulley 1331, and the second advancing wire 1364 and the second retraction wire 1363 may be wound around the second firing drive pulley 1332.

At this time, the first advancing wire 1361 may be wound around the second auxiliary pulley 1352, and the first retraction wire 1362 may be wound around the fourth auxiliary pulley 1354. In addition, the second advancing wire 1364 may be wound around the third auxiliary pulley 1353, and the second retraction wire 1363 may be wound around the first auxiliary pulley 1351.

When a pair of wires wound around one drive pulley are both positioned only on an upper or lower side of the central axis of the shaft 1400, one of the wires may be sharply bent when passing through the auxiliary pulley and winding around the drive pulley.

In other words, when observing the path of the wire entering the auxiliary pulley and exiting toward the drive pulley from the shaft 1400, even when the wire enters the auxiliary pulley parallel to its rotational plane, the wire may be bent at a large angle relative to the rotational plane of the auxiliary pulley when exiting. As a result, significant friction may occur between the wire and the auxiliary pulley.

In the power transmission part 1300 according to an embodiment of the present disclosure, a pair of wires wound around one drive pulley may be separated and respectively positioned on upper and lower sides of the central axis of the shaft 1400 by the arrangement of the auxiliary pulley 1350, thereby reducing the force applied from the wires 1360 to the auxiliary pulley 1350 and decreasing the friction generated between the wires 1360 and the auxiliary pulley 1350.

In some embodiments, in the power transmission part 1300 according to an embodiment of the present disclosure, the first auxiliary pulley 1351, the second auxiliary pulley 1352, the third auxiliary pulley 1353, and the fourth auxiliary pulley 1354 are axially coupled to one rotation shaft, so that forces applied to the center auxiliary pulley rotation shaft 13141 may partially cancel each other out.

For example, the first advancing wire 1361 and the second advancing wire 1364 may be redirected to move in opposite directions along a Y-axis, and the first retraction wire 1362 and the second retraction wire 1363 may also be redirected to move in opposite directions along the Y-axis. As a result, Y-axis components of the forces applied to the center auxiliary pulley rotation shaft 13141 may cancel each other out.

Similarly, a pair of yaw wires 1365 and 1366 wound around the yaw drive pulley 1333 may extend toward the shaft 1400 respectively through yaw auxiliary pulleys 1355 and 1356. In addition, a pair of pitch wires 1367 and 1368 wound around the pitch drive pulley 1334 may extend toward the shaft 1400 respectively through pitch auxiliary pulleys 1357 and 1358.

The yaw auxiliary pulleys 1355 and 1356 and the pitch auxiliary pulleys 1357 and 1358 may be respectively coupled to different shafts.

For example, the yaw auxiliary pulleys 1355 and 1356 may be provided as a pair and spaced apart from each other, and may be disposed at a predetermined angle such that they are not parallel to each other. That is, the pair of yaw auxiliary pulleys 1355 and 1356 may become closer to each other as they extend farther from the yaw drive pulley 1333.

Similarly, the pitch auxiliary pulleys 1357 and 1358 may be provided as a pair and spaced apart from each other, and may be disposed at a predetermined angle such that they are not parallel to each other. That is, the pair of pitch auxiliary pulleys 1357 and 1358 may become closer to each other as they extend farther from the pitch drive pulley 1334.

The yaw auxiliary pulleys 1355 and 1356 and the pitch auxiliary pulleys 1357 and 1358 may be partially received in auxiliary pulley receiving grooves 13143 formed at a lower end of the auxiliary pulley support member 1314. The yaw auxiliary pulleys 1355 and 1356 and the pitch auxiliary pulleys 1357 and 1358 may also be partially received in the auxiliary pulley receiving grooves 13123a formed in the second pulley frame 1312. In addition, auxiliary pulley rotation shafts 1342, 1343, 1344, and 1345 may be fixed e rotation shaft fixing grooves 13123b.

The auxiliary pulley unit may be disposed on the auxiliary pulley support member 1314. The auxiliary pulley support member 1314 may include an opposing surface 1314a facing the second pulley frame 1312.

The auxiliary pulley support member 1314 may include a center auxiliary pulley fixing part 13144 to which the center auxiliary pulley CP is fixed, and a wire guide part 13142 configured to guide the wire 1360 to the center auxiliary pulley fixing part 13144. The auxiliary pulley support member 1314 may also include the auxiliary pulley receiving grooves 13143, in which portions of the yaw auxiliary pulleys 1355 and 1356 and the pitch auxiliary pulleys 1357 and 1358 are received, at a lower end of the wire guide part 13142.

As described above, the surgical instrument 1000 may obtain information on an operational state of the end tool 1100 and control the end tool 1100 to operate within predetermined driving conditions based on the obtained information. Various methods may be employed in the surgical instrument 1000 to obtain information on the operational state of the end tool 1100.

First, a sensor for detecting the operational state may be directly installed in the end tool 1100. For example, during a tissue clamping operation by the end tool 1100, a strain gauge or a load cell may be disposed in the end tool 1100 to directly measure a clamping force of the end tool 1100.

When a sensor is directly installed in the end tool 1100 to detect the operational state as described above, spatial arrangement efficiency within the small-sized end tool 1100 may be reduced, and it may be practically difficult to implement a separate communication structure for communication with the sensor.

Alternatively, the surgical instrument 1000 may estimate the operational state of the end tool 1100 based on an output of the motor of the power generation part 1500. The end tool 1100 of the surgical instrument 1000 may operate by receiving power generated by the motor of the power generation part 1500, and an output value of the motor may be preset in response to the operation of the end tool 1100. Accordingly, the surgical instrument 1000 may estimate the operational state of the end tool 1100 through the output of the motor, such as a current value.

For example, in relation to a rotational motion of the end tool 1100, the end tool 1100 may be configured to rotate within an angular range of −90° to 90° when power corresponding to a motor current value of 1 A is transmitted. In addition, the end tool 1100 may be configured not to rotate beyond the above angular range even when the motor current exceeds 1 A, thereby preventing excessive rotation of the end tool 1100 and resulting damage.

As another example, in relation to a clamping motion of the end tool 1100, the end tool 1100 may be configured to apply a force of 300 N or less to tissue when power corresponding to a motor current value of 1 A is transmitted. Similarly, the end tool 1100 may be configured not to clamp with a force exceeding the above range even when the motor current exceeds 1 A, thereby preventing damage to the end tool 1100 or the tissue.

The surgical instrument 1000 may estimate whether the end tool 1100 is operating within preset driving conditions based on the current value of the motor.

However, when the operational state of the end tool 1100 is estimated based on the output of the motor as described above, there is a problem in that the accuracy of the estimated data cannot be guaranteed due to various factors, such as gear efficiency depending on a gear ratio in a gearbox connected to the motor, wear and aging of the motor or other components, and computational errors in software.

In some embodiments, the surgical instrument 1000 of the present disclosure may obtain information on the operational state of the end tool 1100 by utilizing the structure of the power generation part 1500 that generates power and/or the power transmission part 1300 that transmits power to the end tool 1100.

For example, when the power transmission part 1300 transmits power to the end tool 1100, a predetermined member of the power transmission part 1300 may be subjected to a force by the power, or the member may move or deform. Alternatively, when the end tool 1100 operates beyond the preset driving conditions, the member may be damaged.

The surgical instrument 1000 of the present disclosure may generate sensing data used to determine the extent of operation of the end tool 1100 by installing the sensor part 1600 on the target member provided in one of the power transmission part 1300 and the power generation part 1500, and detecting a force applied to the target member, or detecting movement, deformation, or damage of the target member. In addition, the surgical instrument 1000 may derive information on the operational state of the end tool 1100 in the control part 1700 based on the sensing data generated by the sensor part 1600. The control part 1700 may determine and control whether the end tool 1100 is operating within the preset driving conditions.

As described above, a predetermined member that undergoes changes according to the operational state of the end tool 1100 and is detected by the sensor part 1600 is defined as a “target member.” In other words, the sensor part 1600 may generate sensing data by sensing a force applied to the target member, or by sensing movement, deformation, or damage of the target member. The sensing data thus obtained may be used by the control part 1700 to obtain information on the operational state of the end tool 1100 and to control the operation of the end tool 1100.

In an embodiment, the sensor part 1600 may generate sensing data by detecting changes in the target member that supports a portion of the wire 1360. The control part 1700 may obtain tension information of the wire 1360, which transmits power to the end tool 1100, based on the sensing data.

Since the end tool 1100 receives power through the wire 1360 of the power transmission part 1300, the driving of the end tool 1100 may be associated with tension applied to the wire 1360. When the end tool 1100 operates beyond the driving conditions, a tension greater than a reference value may be applied to the wire 1360. Accordingly, the surgical instrument 1000 may determine and control whether the end tool 1100 is operating within the preset driving conditions by using the sensor part 1600 to detect changes in the target member that supports a portion of the wire 1360, and allowing the control part 1700 to derive tension information of the wire 1360. In this case, the tension information of the wire 1360 derived by the control part 1700 may be continuous numerical data indicating the magnitude of the tension, or discrete judgment data indicating whether the tension exceeds the reference value.

In an embodiment, the sensor part 1600 may be disposed on a direction-changing member that changes a direction in which the wire 1360 extends. In this case, the direction-changing member is defined as the target member, and the sensor part 1600 may detect changes in the direction-changing member to obtain sensing data, based on which the control part 1700 may derive tension information of the wire 1360.

The wire 1360 may be provided with a predetermined tension to transmit power generated by the power generation part 1500 to the end tool 1100. As the direction-changing member changes the direction in which the wire 1360 extends, a predetermined force may be applied to the direction-changing member by the tension of the wire 1360 transmitting the power, and the direction-changing member may move or deform to a certain extent.

Accordingly, the sensor part 1600 may generate sensing data by measuring a force applied to the direction-changing member or by measuring a movement amount, a deformation amount, or the like of the direction-changing member. The control part 1700 may obtain more accurate data by deriving the tension information of the wire 1360 based on the sensing data related to changes in the direction-changing member.

In an embodiment, the sensor part 1600 may obtain sensing data by detecting a force applied to a support member that supports the direction-changing member. When a force is applied to the direction-changing member due to the wire 1360, the support member may also receive a corresponding force from the direction-changing member. Accordingly, the sensor part 1600 may be disposed between the direction-changing member and the support member to measure a force applied to the support member or to measure the movement amount, the deformation amount, or the like of the direction-changing member relative to the support member, thereby obtaining the sensing data. The control part 1700 may obtain tension information of the wire 1360 based on the sensing data.

Hereinafter, the principle of obtaining information on the operational state of the end tool 1100 and controlling the end tool 1100 will be described in detail, focusing on a specific embodiment of the sensor part 1600 disposed in the power transmission part 1300.

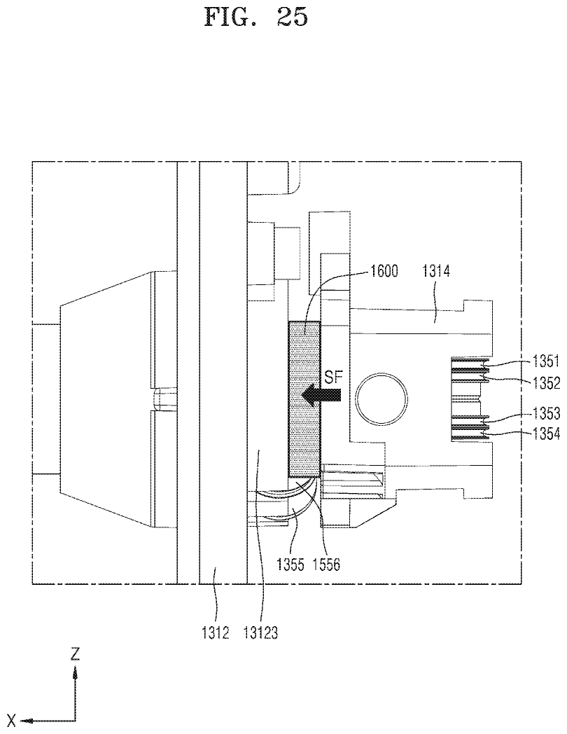

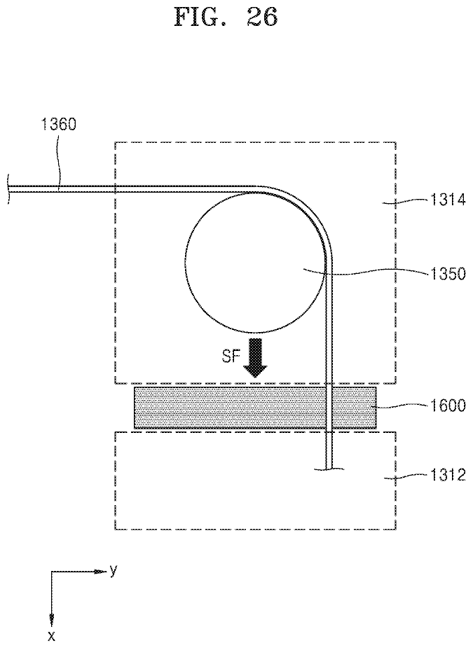

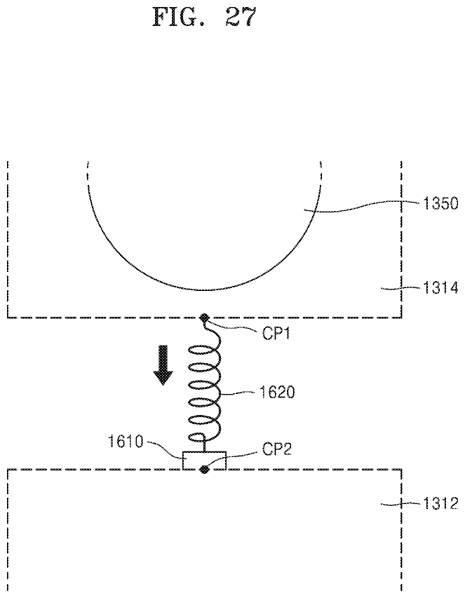

FIG. 25 is a view schematically illustrating the sensor part 1600 disposed in the surgical instrument 1000 of FIG. 1, FIG. 26 is a conceptual view illustrating the power transmission part 1300 and the sensor part 1600 of FIG. 25, and FIG. 27 is a detailed view illustrating the sensor part 1600 of FIG. 25.

Referring to FIGS. 25 to 27, the sensor part 1600 may be positioned between the second pulley frame 1312 and the auxiliary pulley support member 1314.

The sensor part 1600 may obtain sensing data by measuring a force that the auxiliary pulley 1350 or the auxiliary pulley support member 1314, which supports the auxiliary pulley, applies to the second pulley frame 1312. Alternatively, the sensor part 1600 may obtain sensing data by measuring a movement amount of the auxiliary pulley 1350 or the auxiliary pulley support member 1314.

That is, the auxiliary pulley 1350 or the auxiliary pulley support member 1314 supporting the auxiliary pulley may be defined as a target member whose changes are sensed by the sensor part 1600. The sensor part 1600 may detect changes in the auxiliary pulley 1350 or the auxiliary pulley support member 1314 to obtain sensing data, from which the control part 1700 may derive tension information of the wire 1360 and obtain information on the operational state of the end tool 1100.

As described above, the drive pulley 1330 of the power transmission part 1300 may rotate around its axis by receiving power from the power generation part 1500. The wire 1360 may connect the drive pulley 1330 to the end tool 1100, and may be changed in direction by the auxiliary pulley 1350 disposed on the auxiliary pulley support member 1314 between the drive pulley 1330 and the end tool 1100.

As shown in FIG. 20 or the like, the wires 1361, 1362, 1363, and 1364 may respectively extend to the center auxiliary pulleys 1351, 1352, 1353, and 1354 from the drive pulleys 1331 and 1332 disposed on both sides. Accordingly, as illustrated in FIG. 26, forces applied by the wires 1360 to the auxiliary pulley 1350 in the lateral (i.e., Y-axis) direction may cancel each other out, leaving only the force directed toward the second pulley frame 1312.

In other words, the wires 1360 may generate a pressing force SF, which presses the auxiliary pulley 1350 and the auxiliary pulley support member 1314, to which the auxiliary pulley is fixed, against the second pulley frame 1312. Accordingly, the sensor part 1600 may be positioned between the second pulley frame 1312 and the auxiliary pulley support member 1314 to obtain sensing data by measuring the pressing force SF.

In an embodiment, the sensor part 1600 may include a pressure sensor capable of measuring a pressure applied to the second pulley frame 1312. The sensor part 1600 may directly measure the pressure applied to the second pulley frame 1312 to obtain the pressing force SF applied by the auxiliary pulley support member 1314 on the second pulley frame 1312.

In another embodiment, the sensor part 1600 may include a switch member 1610 positioned between the second pulley frame 1312 and the auxiliary pulley support member 1314. The switch member 1610 may be actuated when the auxiliary pulley support member 1314 applies a force to the second pulley frame 1312 or moves relative to the second pulley frame 1312. The sensor part 1600 may obtain sensing data based on the actuation of the switch member 1610.

FIG. 27 illustrates an embodiment in which the switch member 1610 is directly connected to the second pulley frame 1312, but the present disclosure is not limited thereto, and the switch member 1610 may be disposed at various positions between the second pulley frame 1312 and the auxiliary pulley support member 1314, or between the second pulley frame 1312 and the auxiliary pulley 1350.

For example, the sensor part 1600 may further include a connection member 1620 positioned between the auxiliary pulley support member 1314 and the second pulley frame 1312, as shown in FIG. 27. When the auxiliary pulley support member 1314 moves toward or applies a force to the second pulley frame 1312, the connection member 1620 may be deformed, thereby actuating the switch member 1610.

In this case, the connection member 1620 may be elastically deformable. When the auxiliary pulley support member 1314 moves toward or applies a force to the second pulley frame 1312, the connection member 1620 may be elastically compressed, and when the connection member 1620 undergoes elastic deformation exceeding a reference amount, the switch member 1610 may be pressed and actuated. The sensor part 1600 may obtain sensing data on a change of the auxiliary pulley support member 1314 based on the actuation of the switch member 1610.

Alternatively, the sensor part 1600 may obtain sensing data by detecting a change in a first point CP1 of the auxiliary pulley support member 1314 to which the connection member 1620 is connected, or a second point CP2 of the second pulley frame 1312 to which the switch member 1610 is connected. For example, when the auxiliary pulley support member 1314 applies a force to or moves relative to the second pulley frame 1312, the connection member 1620 may be connected to the first point CP1 or the switch member 1610 may be connected to the second point CP2, as shown in FIG. 27.

Alternatively, the sensor part 1600 may obtain sensing data by detecting deformation or damage of the switch member 1610 caused by stress greater than or equal to a preset value applied to the switch member 1610. Stress may be applied to the switch member 1610 when the auxiliary pulley support member 1314 applies a force to or moves relative to the second pulley frame 1312. When the stress greater than or equal to the preset value is applied, the switch member 1610 may be deformed or damaged, and the sensor part 1600 may obtain sensing data regarding such deformation or damage.

In another embodiment, the sensor part 1600 may detect contact between the auxiliary pulley support member 1314 and the second pulley frame 1312. The sensor part 1600 may obtain sensing data on a change of the auxiliary pulley support member 1314 based on the contact. In this case, the sensor part 1600 may obtain sensing data by directly detecting the contact between the two contact points or by detecting a potential change or the like.

As such, the sensor part 1600 may be implemented in any type or manner capable of detecting a force applied by the auxiliary pulley support member 1314 to the second pulley frame 1312, or detecting movement, deformation, or the like of the auxiliary pulley support member 1314 relative to the second pulley frame 1312.

In summary, the sensor part 1600 may obtain sensing data by measuring the pressing force SF applied by the auxiliary pulley support member 1314 to the second pulley frame 1312, or by measuring a movement amount or deformation amount of the auxiliary pulley 1350 or the auxiliary pulley support member 1314. The control part 1700 may obtain tension information of the wire 1360 based on the sensing data and obtain information on the operational state of the end tool 1100. In addition, the control part 1700 may control the operation of the end tool 1100 based on the information related to the operational state of the end tool 1100.

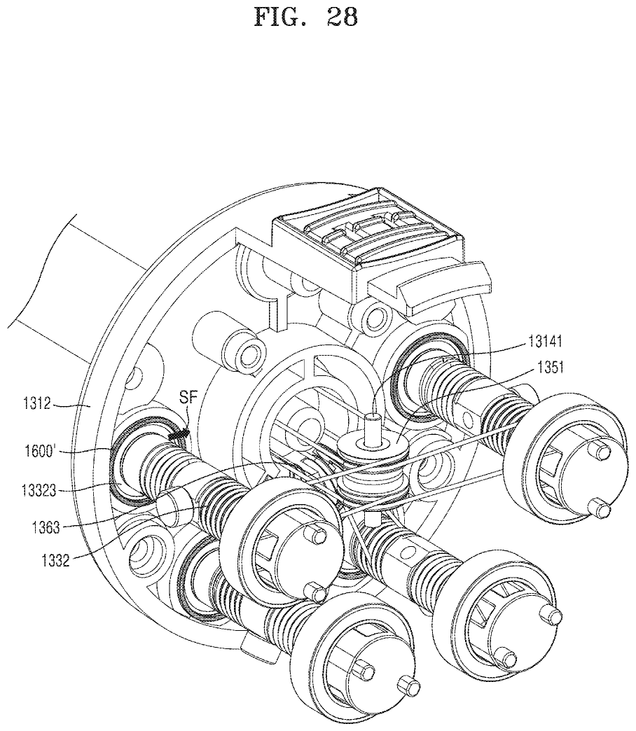

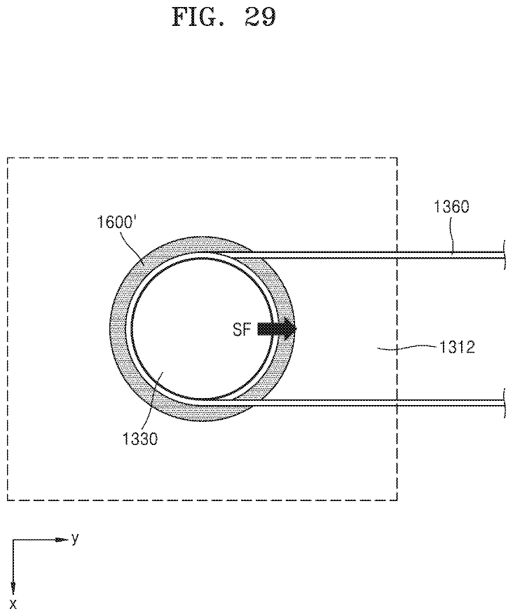

FIG. 28 is a view illustrating another example of the sensor part 1600 disposed in the surgical instrument 1000 of FIG. 1, and FIG. 29 is a conceptual view illustrating the power transmission part 1300 and a sensor part 1600′ of FIG. 28.

Referring to FIGS. 28 and 29, the sensor part 1600′ may be positioned between the second pulley frame 1312 and the drive pulley 1330.

The sensor part 1600′ may measure a force applied by the drive pulley 1330 to the second pulley frame 1312 or a movement amount of the drive pulley 1330, thereby measuring a force applied by the wire 1360 to the drive pulley 1330. In this case, the drive pulley 1330 may be defined as a target member whose change is sensed by the sensor part 1600′. The sensor part 1600′ may generate sensing data based on the force applied by the wire 1360 to the drive pulley 1330, and the control part 1700 may derive tension information of the wire 1360 based on the sensing data.

As described above, the drive pulley 1330 of the power transmission part 1300 may receive power from the power generation part 1500 and rotate around its axis. The wire 1360 may connect the drive pulley 1330 to the end tool 1100 and may be wound around the drive pulley 1330. The wire 1360 may enter and exit the drive pulley 1330 via the auxiliary pulley 1350. Accordingly, as shown in FIG. 29, forces applied by the wires 1360 to the drive pulley 1330 in a vertical direction, i.e., the Z-axis direction, may cancel each other out, leaving only a force directed toward the auxiliary pulley 1350.

The drive pulley 1330 may be positioned such that one end is inserted into the second drive pulley coupling part 13126 of the second pulley frame 1312, and another end is inserted into the first drive pulley coupling part 13112 of the first pulley frame 1311. Accordingly, a force may be applied to the drive pulley 1330 toward the auxiliary pulley 1350 by the wire 1360, and the drive pulley 1330 may apply the pressing force SF to the first pulley frame 1311 and the second pulley frame 1312. The sensor part 1600′ may be positioned between the drive pulley 1330 and the first pulley frame 1311, or between the drive pulley 1330 and the second pulley frame 1312, and may obtain sensing data by measuring the pressing force SF.

FIGS. 28 and 29 illustrate an embodiment in which the sensor part 1600′ is positioned between the drive pulley 1330 and the second pulley frame 1312. For example, the sensor part 1600′ may be positioned on the second drive pulley coupling part 13126 of the second pulley frame 1312. In the description focusing on the second firing drive pulley 1332, the sensor part 1600′ may be formed in a ring shape surrounding a pulley bearing 13323 at the second drive pulley coupling part 13126, as shown in FIG. 28. However, the position and shape of the sensor part 1600′ are not particularly limited.

In some embodiments, as described above, the sensor part 1600′ may measure a pressure applied to the second pulley frame 1312, or detect a force applied to the second pulley frame 1312 based on, for example, the actuation of the switch member, deformation of the connection member, or the like. Alternatively, the sensor part 1600′ may detect contact or measure changes in current to detect a minute amount of movement or deformation of the drive pulley 1330. Specific embodiments of the detailed structure of the sensor part 1600′ may refer to the descriptions previously provided for the sensor part 1600 of FIG. 27.

As such, the sensor part 1600′ may be implemented in any type or manner capable of detecting a force applied by the drive pulley 1330 to the first pulley frame 1311 or the second pulley frame 1312, or detecting movement or deformation of the drive pulley 1330 relative to the first pulley frame 1311 or the second pulley frame 1312.

In summary, the sensor part 1600′ may obtain sensing data by measuring the pressing force SF applied by the drive pulley 1330 to the first pulley frame 1311 or the second pulley frame 1312, or by measuring a predetermined amount of movement or deformation of the drive pulley 1330. The control part 1700 may obtain tension information of the wire 1360 based on the sensing data and obtain information related to the operational state of the end tool 1100. In addition, the control part 1700 may control the operation of the end tool 1100 based on the information on the operational state of the end tool 1100.

As described above, a direction-changing member, such as the drive pulley 1330 or the auxiliary pulley 1350, which is disposed in the power transmission part 1300 and capable of changing the direction in which the wire 1360 extends, may be defined as a target member. The sensor part 1600 may obtain sensing data by detecting a force applied to the direction-changing member or detecting movement or deformation of the direction-changing member.

The control part 1700 may derive tension information of the wire 1360 based on the sensing data and obtain information on the operational state of the end tool 1100. In addition, the control part 1700 may control the end tool 1100 to operate within a preset driving range based on the information on the operational state of the end tool 1100.

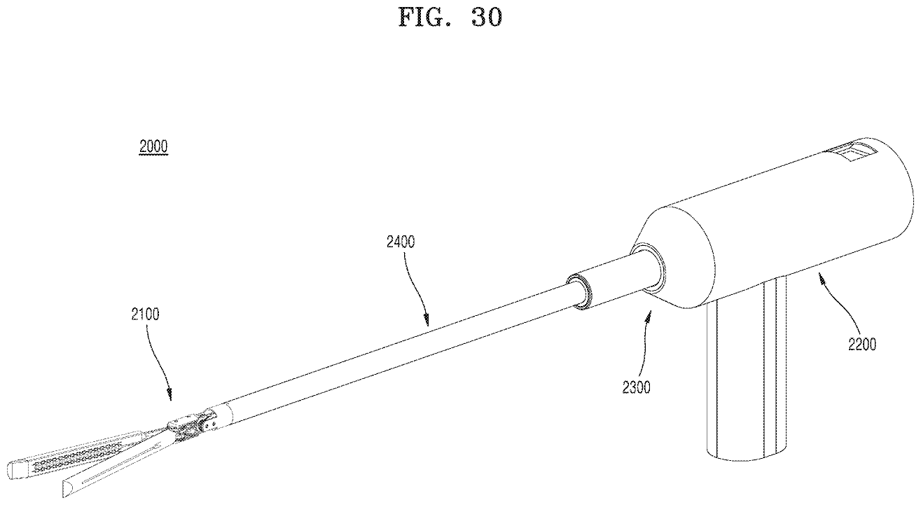

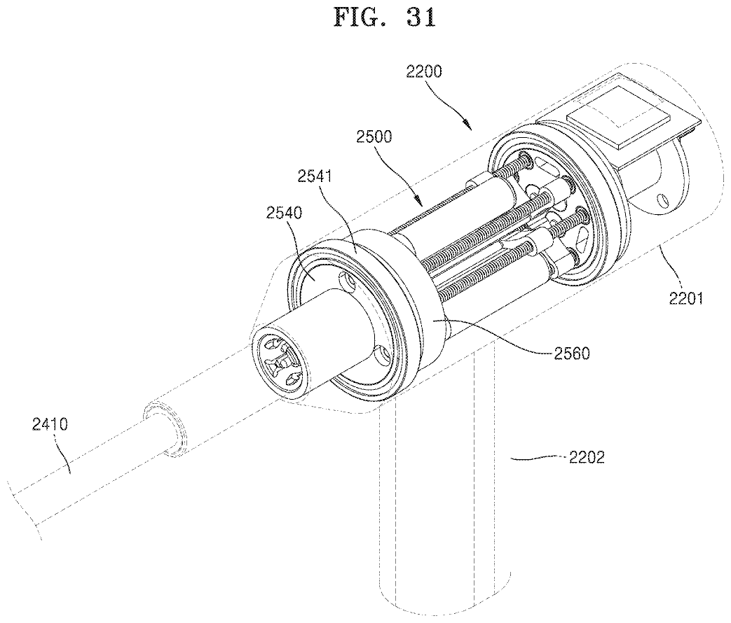

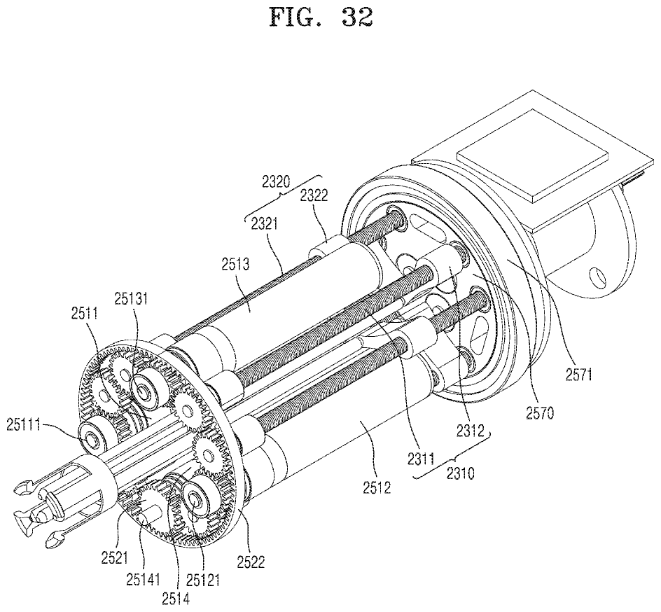

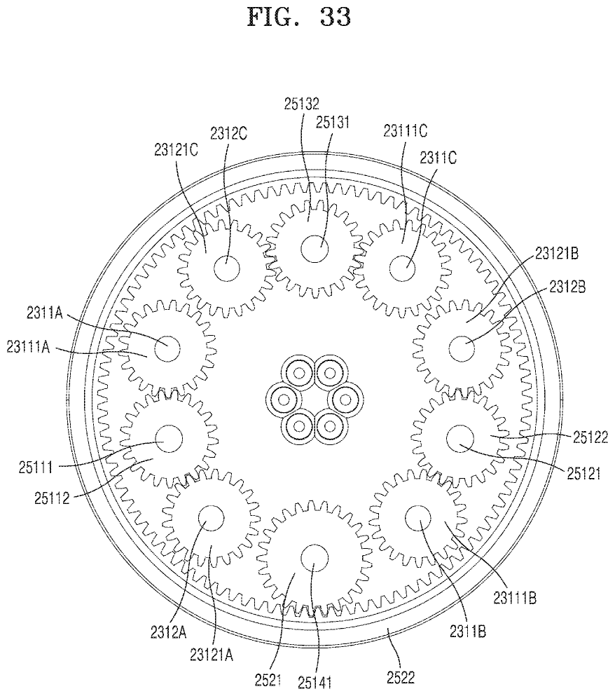

FIG. 30 is a perspective view illustrating a surgical instrument 2000 according to another embodiment of the present disclosure. FIG. 31 is a perspective view illustrating a manipulation part 2200 and a power generation part 2500 of the surgical instrument of FIG. 30, FIG. 32 is a magnified perspective view illustrating the power generation part 2500 of FIG. 31, and FIG. 33 is a cross-sectional view illustrating a gear structure of the power generation part 2500 of FIG. 31.

The surgical instrument 2000 of FIG. 30 is different from the surgical instrument 1000 of FIG. 1 in the structure and power transmission method of a power transmission part 2300 and the power generation part 2500, and thus the following description will focus on these differences, and for other components such as an end tool, reference will be made to the descriptions provided for the surgical instrument 1000 of FIG. 1.

Referring to FIGS. 30 to 33, the surgical instrument 2000 may include the power generation part 2500 that generates power to control an end tool 2100.

The power generation part 2500 may be disposed to be at least partially accommodated in a housing 2201 of the manipulation part 2200. Here, the housing 2201 may refer to a component that forms an outer shape of the manipulation part 2200, but in the case of a drive module for the surgical instrument 2000, the housing 2201 may refer to a component that forms an outer shape of a module body.

When a user manipulates the manipulation part 2200, the power generation part 2500 may generate power to control the end tool 2100 based on the manipulation.

The power generation part 2500 may include a motor pack 2510 including at least one motor.

The motor pack 2510 may include at least one motor. For example, the motor pack 2510 may include at least one motor that generates power to drive the end tool 2100 based on a signal input to the manipulation part 2200.

The motor pack 2510 may include a yaw drive motor 2511. The yaw drive motor 2511 may generate power to yaw-rotate the end tool 2100. For example, the yaw drive motor 2511 may generate a driving force for yaw rotation of the end tool 2100 when a user manipulates the manipulation part 2200 to yaw-rotate the end tool 2100.

The driving force generated by the yaw drive motor 2511 may be transmitted to the power transmission part 2300 to move yaw drive wires (not shown), thereby allowing the end tool 2100 to yaw-rotate.

The yaw drive motor 2511 may include a yaw motor rotation shaft 25111 formed to extend in one direction. The yaw motor rotation shaft 25111 is a part that rotates when the yaw drive motor 2511 starts driving. For example, the yaw motor rotation shaft 25111 may be formed to extend in a direction toward the power transmission part 2300 from a body of the yaw drive motor 2511. As the yaw motor rotation shaft 25111 rotates, the yaw drive wires (not shown) may move, and as a result, the end tool 2100 may perform a yaw rotation or an actuation motion.

The motor pack 2510 may include a pitch drive motor 2512. The pitch drive motor 2512 may generate power to pitch-rotate the end tool 2100. For example, the pitch drive motor 2512 may generate a driving force for pitch rotation of the end tool 2100 when a user manipulates the manipulation part 2200 to pitch-rotate the end tool 2100.

The driving force generated by the pitch drive motor 2512 may be transmitted to the power transmission part 2300 to move pitch drive wires (not shown), thereby enabling the pitch rotation of the end tool 2100.

The pitch drive motor 2512 may include a pitch motor rotation shaft 25121 formed to extend in one direction. The pitch motor rotation shaft 25121 is a part that rotates when the pitch drive motor 2512 starts driving. For example, the pitch motor rotation shaft 25121 may be formed to extend in a direction toward the power transmission part 2300 from a body of the pitch drive motor 2512. As the pitch motor rotation shaft 25121 rotates, the pitch drive wires (not shown) may move, and as a result, the end tool 2100 may pitch-rotate.

The motor pack 2510 may include a firing drive motor 2513. The firing drive motor 2513 may generate power to linearly move an operation member (not shown) provided in the end tool 2100. For example, the firing drive motor 2513 may generate a driving force to linearly move the operation member (not shown) when a user manipulates the manipulation part 2200 to linearly move the operation member (not shown) of the end tool 2100.

The driving force generated by the firing drive motor 2513 may be transmitted to the power transmission part 2300 to move firing drive wires (not shown), thereby enabling the operation member (not shown) to move in a cartridge 2150 of the end tool 2100.

The firing drive motor 2513 may include a firing motor rotation shaft 25131 formed to extend in one direction. The firing motor rotation shaft 25131 is a part that rotates when the firing drive motor 2513 is driven. For example, the firing motor rotation shaft 25131 may be formed to extend in a direction toward the power transmission part 2300 from a body of the firing drive motor 2513. As the firing motor rotation shaft 25131 rotates, the firing drive wires (not shown) may move, and as a result, in the end tool 2100, the operation member (not shown) linearly moves to perform stapling and cutting motions.

The power generation part 2500 may include a roll drive motor 2514 that generates power to roll-rotate the motor pack 2510. For example, the roll drive motor 2514 may generate a driving force to roll-rotate the motor pack 2510 when a user manipulates the manipulation part 2200 to rotate the motor pack 2510.

In an embodiment, the roll drive motor 2514 may be included in the motor pack 2510. For example, the roll drive motor 2514 may be provided inside the motor pack 2510 together with the yaw drive motor 2511 and the pitch drive motor 2512.

The roll drive motor 2514 may be driven by the user's manipulation to generate a driving force to rotate the motor pack 2510. In this case, the roll drive motor 2514 may move together with the motor pack 2510. For example, when the motor pack 2510 is rotated by the roll drive motor 2514, the roll drive motor 2514, as a component provided in the motor pack 2510, may rotate together with the motor pack 2510.

In another embodiment, the roll drive motor 2514 may not be provided in the motor pack 2510. For example, the roll drive motor 2514 may be positioned outside the motor pack 2510.

The roll drive motor 2514 may be driven by the user's manipulation to generate a driving force to rotate the motor pack 2510. In this case, the roll drive motor 2514 may move independently from the motor pack 2510. For example, unlike what will be described below, the motor pack 2510 may perform a roll rotation by the roll drive motor 2514, but the roll drive motor 2514 may not rotate together with the motor pack 2510. For example, the yaw drive motor 2511, the pitch drive motor 2512, and the firing drive motor 2513 may rotate together, whereas the roll drive motor 2514 may not rotate together therewith.

Hereinafter, for convenience of description, the roll drive motor 2514 will be described as being included in the motor pack 2510 by way of example, but it will be understood by those skilled in the art that the roll drive motor 2514 may not be included in the motor pack 2510 in the following description. In addition, those skilled in the art will understand that, when the roll drive motor 2514 is not provided in the motor pack 2510, the following content may be appropriately modified and applied.

The roll drive motor 2514, along with a roll drive gear 2521 and a rotation guide unit 2522, which will be described later, may serve as a roll rotation unit 2520 to roll-rotate the motor pack 2510.

The motor pack 2510 may include a base plate 2560. The base plate 2560 may be positioned in front of the yaw drive motor 2511, the pitch drive motor 2512, the roll drive motor 2514, and the firing drive motor 2513.

At least one through hole may be formed in the base plate 2560. For example, at least as many through holes as the number of motors included in the motor pack 2510 may be formed in the base plate 2560. The through hole is a part through which the rotation shaft of the respective motor passes.

For example, the yaw motor rotation shaft 25111 extends from the body of the yaw drive motor 2511, and may be formed to extend through the base plate 2560. In addition, the pitch motor rotation shaft 25121 extends from the body of the pitch drive motor 2512, and may be formed to extend through the base plate 2560. In addition, the firing motor rotation shaft 25131 extends from the body of the firing drive motor 2513, and may be formed to extend through the base plate 2560. In addition, a roll motor rotation shaft 25141 extends from a body of the roll drive motor 2514, and may be formed to extend through the base plate 2560.

In an embodiment, the power generation part 2500 may further include a bearing plate 2540 and a first bearing 2541. The bearing plate 2540 and the first bearing 2541 may reduce rotational friction between the motor pack 2510 and the housing 2201 when the motor pack 2510 roll-rotates.

The bearing plate 2540 may be positioned in front of the rotation guide unit 2522. For example, at least one through hole may be formed in the bearing plate 2540. The through hole is a part through which the rotation shaft of the respective motor passes.

The first bearing 2541 may be positioned on an outer circumferential surface of the bearing plate 2540. For example, the first bearing 2541 may be positioned to cover the outer circumferential surface of the bearing plate 2540. Thus, when the motor pack 2510 roll-rotates, the bearing plate 2540 rotates together with the motor pack 2510, and in this case, the bearing plate 2540 and the first bearing 2541 may reduce the rotational friction between the motor pack 2510 and the housing 2201.

In an embodiment, the power generation part 2500 may further include a circuit plate 2570 and a second bearing 2571. The circuit plate 2570 and the second bearing 2571 may reduce the rotational friction between the motor pack 2510 and the housing 2201 when the motor pack 2510 roll-rotates.

The circuit plate 2570 may be positioned at the rear of the motor pack 2510. As will be described later, the circuit plate 2570 is a part to which a circuit unit (not shown) is connected.

The circuit plate 2570 is connected to the motor pack 2510 and may rotate in response to the rotation of the motor pack 2510. In addition, the second bearing 2571 may be positioned on an outer circumferential surface of the circuit plate 2570. Thus, when the motor pack 2510 roll-rotates, the circuit plate 2570 rotates together with the motor pack 2510, and in this case, the circuit plate 2570 and the second bearing 2571 may reduce the rotational friction between the motor pack 2510 and the housing 2201.

The surgical instrument 2000 according to an embodiment of the present disclosure may further include the circuit unit (not shown) for controlling the power generation part 2500. The circuit unit (not shown) may include an electronic circuit for controlling the operation of the motor pack 2510. The circuit unit (not shown) may include a motor driver, a motor controller, a microcontroller unit, and the like, but is not limited thereto, and may have various other components capable of driving the motor pack 2510.

In an embodiment, the surgical instrument may further include at least one sub-circuit unit (not shown). The sub-circuit unit may preprocess various signals for controlling the motor pack 2510. In addition, the sub-circuit unit may transmit the preprocessed signals to the circuit unit (not shown).

In the above, the principle of power generation of the surgical instrument 2000 according to an embodiment of the present disclosure and the driving of the motor pack 2510 have been described. For example, as described above, when power is generated by the power generation part 2500 of the surgical instrument 2000 of the present disclosure, the wire may move along the pulleys and transmit the power to the end tool 2100. Accordingly, the end tool 2100 may perform a yaw motion, a pitch motion, an actuation motion, and a firing motion. Hereinafter, the principle of power transmission of the surgical instrument 2000 will be described in detail, with a focus on the configuration of the power transmission part 2300.

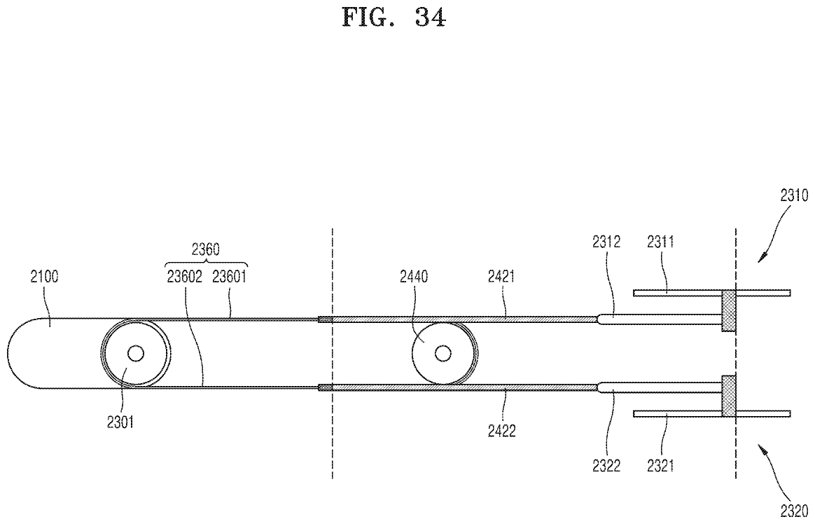

FIGS. 34 to 36 are conceptual diagrams illustrating a power transmission structure of the surgical instrument 2000 of FIG. 30 and operations of the end tool 2100.

Referring to FIGS. 34 to 36, the surgical instrument 2000 may include drive wires 2360.

The drive wires 2360 may control the rotation of the end tool 2100. The drive wires 2360 connects the end tool 2100 to the power transmission part 2300, enabling the power generated by the power generation part 2500 to be transmitted to the end tool 2100.

In the present specification, the drive wires 2360 may refer to all wires connected to the end tool 2100 that are capable of controlling the operation of the end tool 2100. For example, the drive wires 2360 may refer to the yaw drive wires (not shown), which move by the driving of the yaw drive motor 2511 and enable yaw rotation of the end tool 2100. Alternatively, the drive wires 2360 may refer to the pitch drive wires (not shown), which move by the driving of the pitch drive motor 2512 and enable pitch rotation of the end tool 2100. Alternatively, the drive wires 2360 may refer to the firing drive wires (not shown), which move by the driving of the firing drive motor 2513 and enable the operation member (not shown) of the end tool 2100 to move.

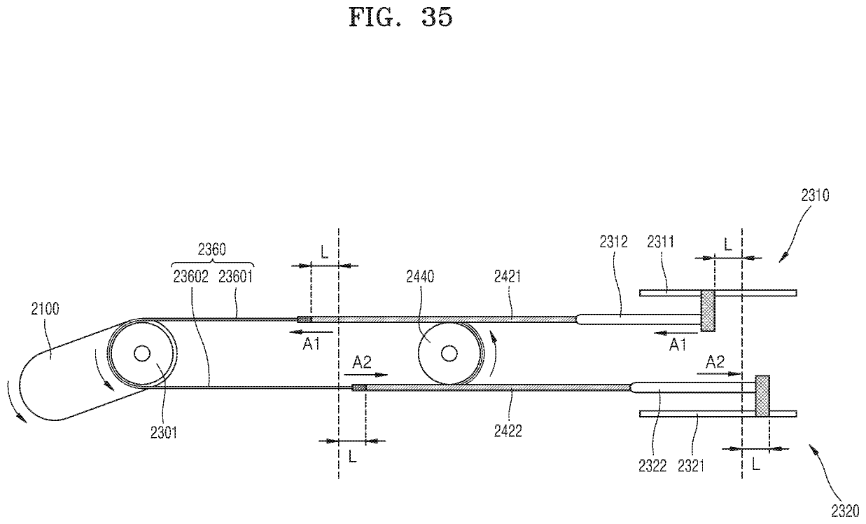

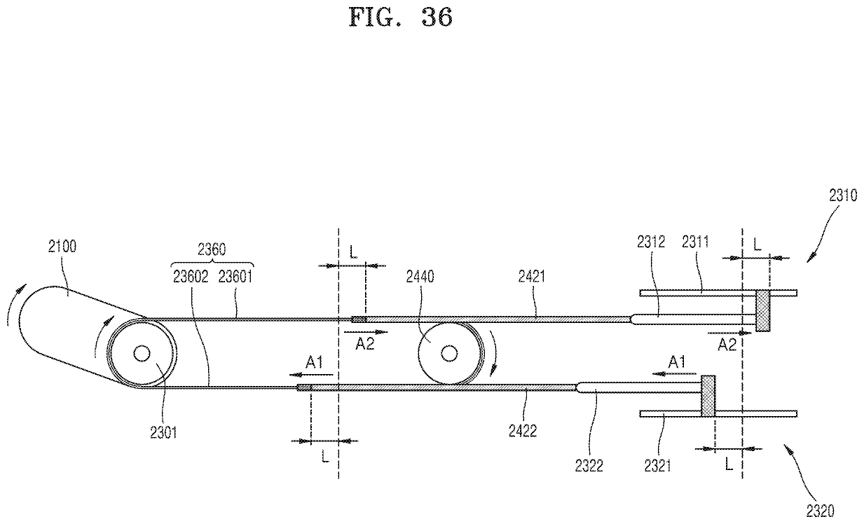

FIGS. 35 and 36 illustrate operational states in which the end tool 2100 rotates in a specific direction. However, the operation of the end tool 2100, based on power transmission in the surgical instrument 2000, is not limited thereto and may also include a linear movement of the operation member (not shown), which may be controlled by the movement of the drive wire 2360.

The drive wires 2360 may include a first wire 23601 and a second wire 23602. As described above, the yaw drive wires, the pitch drive wires, and the firing drive wires may each be provided as a pair. That is, the drive wires 2360 may include a pair of wires, which are the first wire 23601 and the second wire 23602, and the pair of wires may move to control the operation of the end tool 2100.

The first wire 23601 and the second wire 23602 may be provided as a single wire. Both strands of the drive wire 2360 centered on an intermediate point of the drive wire 2360, which is a single wire, may be referred to as the first wire 23601 and the second wire 23602, respectively. Alternatively, the first wire 23601 and the second wire 23602 may also be formed as separate wires and connected by a coupling member (not shown).

The drive wires 2360 may move as the first wire 23601 and the second wire 23602, which are wound around a drive pulley 2301, are pulled and released. The drive pulley 2301 may guide the movement of the first wire 23601 and the second wire 23602. In addition, the power transmission part 2300 of the present disclosure may include one or more sub-drive pulleys (not shown) to control the movement path and direction of the drive wire 2360.

For example, as shown in FIG. 35, as the first wire 23601 moves in one direction (an arrow A1 direction) and the second wire 23602 moves in another direction (an arrow A2 direction), the end tool 2100 may rotate in the counterclockwise direction on the plane of the drawing. In some embodiments, as shown in FIG. 36, as the second wire 23602 moves in one direction (the arrow A1 direction) and the first wire 23601 moves in another direction (the arrow A2 direction), the end tool 2100 may rotate in the clockwise direction on the plane of the drawing.

That is, as one wire of the first wire 23601 and the second wire 23602 is pulled in another direction (the arrow A2 direction), the end tool 2100 may rotate toward the one wire. Accordingly, the power transmission part 2300 may control the operation direction and magnitude of the end tool 2100 by controlling the pulling direction and length of the drive wire 2360. At this time, the power transmission part 2300 may control a single operation by controlling the pair of drive wires 23601 and 23602 to move in opposite directions.

The power transmission part 2300 may include a first power transmission unit 2310 and a second power transmission unit 2320.

The first power transmission unit 2310 is connected to the first wire 23601, and may control the movement of the first wire 23601. The first power transmission unit 2310 may move linearly in a longitudinal direction by the driving of the motor and control the movement of the first wire 23601.

The first power transmission unit 2310 may include a first lead screw 2311 and a first linear movement guide 2312.

For example, the first power transmission unit 2310 may cause the first lead screw 2311 to rotate by the driving of the motor, thereby allowing the first linear movement guide 2312 to linearly move. A first wire coupling member 2421 of a connection part 2400 to be described later may be coupled to one end of the first wire 23601. Accordingly, as the first linear movement guide 2312 linearly moves to push the first wire coupling member 2421, the first wire coupling member 2421 also moves linearly, thereby moving the first wire 23601.

The second power transmission unit 2320 is connected to the second wire 23602 and may control the movement of the second wire 23602. The second power transmission unit 2320 may move linearly in the longitudinal direction by the driving of the motor and control the movement of the second wire 23602.

The second power transmission unit 2320 may include a second lead screw 2321 and a second linear movement guide 2322.

For example, the second power transmission unit 2320 may cause the second lead screw 2321 to rotate by the driving of the motor, thereby allowing the second linear movement guide 2322 to linearly move. A second wire coupling member 2422 of the connection part 2400 to be described later may be coupled to one end of the second wire 23602. Accordingly, as the second linear movement guide 2322 linearly moves to push the second wire coupling member 2422, the second wire coupling member 2422 also moves linearly while remaining in contact with the second linear movement guide 2322, thereby moving the second wire 23602.

The connection part 2400 may include the first wire coupling member 2421 and the second wire coupling member 2422.

The first wire coupling member 2421 may move linearly with one end connected to the first wire 23601 and another end remaining in contact with the first linear movement guide 2312. When the first linear movement guide 2312 moves linearly by the driving of the motor, the first wire coupling member 2421 may be pushed by the first linear movement guide 2312 and may move linearly. The first wire 23601 connected to the first wire coupling member 2421 may also move linearly in response to the movement of the first wire coupling member 2421.

The second wire coupling member 2422 may move linearly with one end connected to the second wire 23602 and another end remaining in contact with the second linear movement guide 2322. When the second linear movement guide 2322 moves linearly by the driving of the motor, the second wire coupling member 2422 may be pushed by the second linear movement guide 2322 and may move linearly. The second wire 23602 connected to the second wire coupling member 2422 may also move linearly in response to the movement of the second wire coupling member 2422.

The connection part 2400 may further include a direction-changing part 2440. The direction-changing part 2440 may switch movement directions between the first wire 23601 and the second wire 23602.

The first wire 23601 and the second wire 23602 may extend while being wound around the drive pulley 2301. At this time, one end of the first wire 23601 may be coupled to the first wire coupling member 2421, and one end of the second wire 23602 may be coupled to the second wire coupling member 2422. When the first linear movement guide 2312 moves linearly in one direction (the arrow A1 direction) while remaining in contact with the first wire coupling member 2421 by the driving of the motor, the first linear movement guide 2312 may push the first wire coupling member 2421, thereby allowing the first wire 23601 to move in one direction (the arrow A1 direction) together with the first wire coupling member 2421.

In some embodiments, since the second wire coupling member 2422 is only in face-to-face contact with the second linear movement guide 2322, the second linear movement guide 2322 can push the second wire coupling member 2422 in one direction (the arrow A1 direction) by a certain distance L, but cannot pull the second wire coupling member 2422 in another direction (the arrow A2 direction) by the same distance L. That is, when the first linear movement guide 2312 moves the first wire coupling member 2421 in one direction (the arrow A1 direction), the second wire coupling member 2422 cannot move in another direction (the arrow A2 direction) correspondingly. When the second wire 23602 fails to move in another direction (the arrow A2 direction) by the same distance L that the first wire 23601 has moved in one direction, a tension of the drive wire 2360 may become excessively high or low, which may cause the end tool 2100 to be unable to operate normally.

Similarly, when the second linear movement guide 2322 moves linearly in one direction (the arrow A1 direction) while remaining in contact with the second wire coupling member 2422, since the first wire coupling member 2421 is only in face-to-face contact with the first linear movement guide 2312, the first linear movement guide 2312 cannot pull the first wire coupling member 2421 in another direction (the arrow A2 direction) by the same distance L. When the first wire 23601 fails to move in another direction (the arrow A2 direction) by the same distance L that the second wire 23602 has moved in one direction, the tension of the drive wire 2360 may become excessively high or low, which may cause the end tool 2100 to be unable to operate normally.

Accordingly, in the surgical instrument 2000 of the present disclosure, the connection part 2400 further includes the direction-changing part 2440, allowing the first wire 23601 and the second wire 23602 to move in opposite directions by corresponding distances, thereby maintaining the tension of the drive wire 2360 and enabling the normal operation of the end tool 2100.

For example, the direction-changing part 2440 may be a pulley disposed between the first wire coupling member 2421 and the second wire coupling member 2422, or gears that are respectively engaged with the first wire coupling member 2421 and the second wire coupling member 2422. The direction-changing part 2440 may be provided with any type or form of component capable of reversing movement directions of the first wire coupling member 2421 and the second wire coupling member 2422.

Specifically, as shown in FIG. 35, when the first linear movement guide 2312 moves in one direction (the arrow A1 direction) by the driving of the motor while pushing the first wire coupling member 2421, the first wire 23601 can move in one direction (the arrow A1 direction). When the first wire 23601 moves in one direction (the arrow A1 direction), the second wire 23602 may move in another direction (the arrow A2 direction) by the same distance L due to the direction-changing part 2440. As a result, the drive wires 2360 may maintain the tension while transmitting power to the end tool 2100, which allows the end tool 2100 to rotate toward the second wire 23602.

Similarly, as shown in FIG. 36, when the second linear movement guide 2322 moves in one direction (the arrow A1 direction) by the driving of the motor while pushing the second wire coupling member 2422, the second wire 23602 can move in one direction (the arrow A1 direction). When the second wire 23602 moves in one direction (the arrow A1 direction), the first wire 23601 may move in another direction (the arrow A2 direction) by the same distance L due to the direction-changing part 2440. As a result, the drive wires 2360 may maintain the tension while transmitting power to the end tool 2100, which allows the end tool 2100 to rotate toward the first wire 23601.

The surgical instrument 2000 may include a plurality of the first power transmission units 2310 and a plurality of the second power transmission units 2320. The first power transmission units 2310 and the second power transmission units 2320 may be provided in each of the yaw drive motor 2511, the pitch drive motor 2512, and the firing drive motor 2513, and may have substantially the same detailed structure to transmit power generated from the power generation part 2500.

Hereinafter, the power transmission method of the first power transmission unit 2310 and the second power transmission unit 2320 will be described in detail, focusing on the firing drive motor 2513 and a gear structure connected thereto.

For example, as illustrated in FIGS. 32 and 33, a main gear 25132 may be disposed in the firing drive motor 2513, and a first sub-gear 23111C and a second sub-gear 23121C may be engaged with and disposed on opposite sides of the main gear 25132. The first lead screw 2311 having threads on an outer surface thereof may be disposed on the first sub-gear 23111C, and the second lead screw 2321 having threads on an outer surface thereof may be disposed on the second sub-gear 23121C.

The first linear movement guide 2312 and the second linear movement guide 2322 may be screw-coupled to the first lead screw 2311 and the second lead screw 2321, respectively. As the first lead screw 2311 rotates, the first linear movement guide 2312 may move linearly in the longitudinal direction. Likewise, as the second lead screw 2321 rotates, the second linear movement guide 2322 may move linearly in the longitudinal direction.

The first linear movement guide 2312 and the second linear movement guide 2322 may come into contact with the first wire coupling member 2421 and the second wire coupling member 2422, respectively, to which the first wire and the second wire are connected. Although not shown in the drawings, the driving wire may maintain tension and have its movement direction adjusted by the direction-changing part. Accordingly, as previously described with reference to FIGS. 34 to 36, when the firing drive motor 2513 is driven, the first power transmission unit 2310 and the second power transmission unit 2320 may move the drive wire 2360.

Likewise, the yaw drive motor 2511 and the pitch drive motor 2512 may also operate in substantially the same manner as the firing drive motor 2513, and the power generated from each motor may be transmitted to the end tool 2100 through the power transmission structure.

As described above, the surgical instrument 2000 includes the power transmission units 2310 and 2320 corresponding to the motors, and may transmit power to the end tool 2100 by linearly moving the drive wire 2360 through the power transmission units 2310 and 2320. For example, the surgical instrument 2000 may include a sensor part configured to obtain sensing data related to the operation degree of the end tool 2100. Hereinafter, the sensor part provided in the surgical instrument 2000 and a method for generating sensing data by the sensor part will be described in detail.

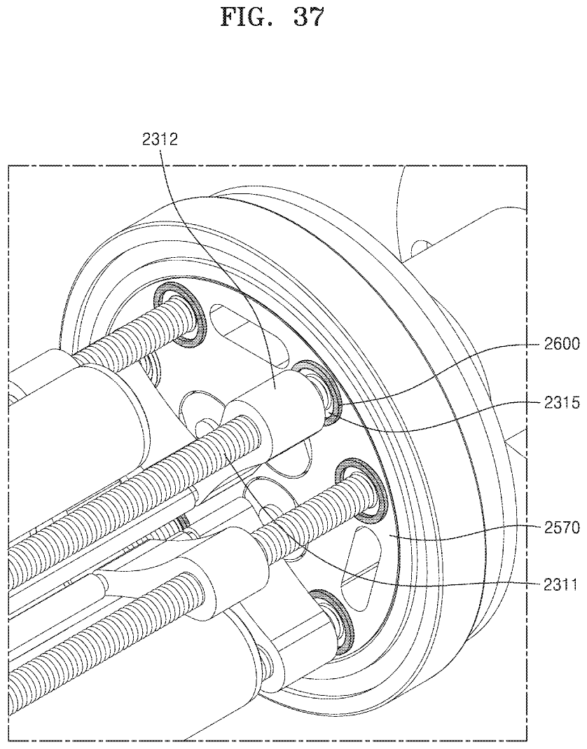

FIG. 37 is a view schematically illustrating a sensor part 2600 disposed in the surgical instrument 2000 of FIG. 30.

Referring to FIG. 37, the surgical instrument 2000 may further include the sensor part 2600.

As described above, the surgical instrument 2000 may include the first power transmission unit 2310 and the second power transmission unit 2320, each configured to linearly move the drive wire 2360 in order to transmit power generated from the power generation part 2500. Hereinafter, for convenience of description, the sensing principle of the sensor part 2600 will be described focusing on the first power transmission unit 2310.

The first power transmission unit 2310 may include the first lead screw 2311 positioned between the bearing plate 2540 and the circuit plate 2570. One end of the first lead screw 2311 may be coupled to the circuit plate 2570, and another end thereof may be coupled to the base plate 2560. That is, the first lead screw 2311 may be fixed between the circuit plate 2570 and the base plate 2560. In this case, the bearing plate 2540 and the circuit plate 2570, to which opposite ends of the first lead screw 2311 are coupled, and the base plate 2560 through which the first lead screw 2311 passes, are all capable of supporting the first lead screw 2311, and are hereinafter collectively referred to as “support plates” in the present specification.

The first lead screw 2311 may be coupled with the first linear movement guide 2312, and the first linear movement guide 2312, while coupled to the first lead screw 2311, may move linearly under motor drive to move the drive wire 2360. At this time, the drive wire 2360 moves with a predetermined tension, thereby controlling the operation of the end tool 2100.

When the first linear movement guide 2312 moves while being coupled to the first lead screw 2311, the first lead screw 2311 may receive a force in the opposite direction due to action and reaction. For example, when the first linear movement guide 2312 moves linearly toward the end tool 2100 and pushes the first wire coupling member 2421 to move the drive wire 2360, the first lead screw 2311 may be subjected to a force in a direction away from the end tool 2100.

The sensor part 2600 may obtain sensing data by sensing a force applied to the first lead screw 2311, and based on this, a control part (not shown) may derive tension information of the drive wire 2360 and obtain information on the operational state of the end tool 2100.

In an embodiment, as illustrated in FIG. 37, the sensor part 2600 may be disposed on the circuit plate 2570 and may sense a force applied to the first lead screw 2311. In this case, the first lead screw 2311 may be defined as a target member whose changes are sensed by the sensor part 2600.

In some embodiments, one end of the first lead screw 2311 may be supported by being coupled to the circuit plate 2570. Accordingly, the sensor part 2600 may be positioned between one end of the first lead screw 2311 and the circuit plate 2570 to detect a force applied to the first lead screw 2311.

Although not specifically illustrated in the drawings, the sensor part 2600 may be positioned between a screw bearing 2315, which is coupled to one end of the first lead screw 2311, and the first lead screw 2311. Alternatively, as shown in FIG. 37, the sensor part 2600 may be positioned between the screw bearing 2315 and the circuit plate 2570. In addition, the sensor part 2600 may be provided in a ring shape as shown in FIG. 37, but its arrangement and shape are not particularly limited.

In some embodiments, as described above, the sensor part 2600 may measure a pressure applied to the circuit plate 2570 or detect a force applied to the circuit plate 2570 based on the deformation of the connection member or the actuation of the switch member. Alternatively, the sensor part 2600 may detect contact directly or measure a change in current or the like to measure a minute amount of movement or deformation of the circuit plate 2570. Specific examples of the detailed structure of the sensor part 2600 may refer to the descriptions above regarding the sensor part 1600 of FIG. 27.

As described above, the sensor part 2600 may be implemented in any type or manner capable of detecting a force applied by the first lead screw 2311 to the circuit plate 2570, or detecting movement, deformation, or the like of the first lead screw 2311 relative to the circuit plate 2570.

In summary, the sensor part 2600 may obtain sensing data by measuring a pressing force applied by the first lead screw 2311 to the circuit plate 2570, or measuring a predetermined amount of movement or deformation of the first lead screw 2311. The control part (not shown) may derive tension information of the drive wire 2360 based on the sensing data, and obtain information on the operational state of the end tool 2100. In addition, the control part (not shown) may control the operation of the end tool 2100 based on the information on the operational state of the end tool 2100.

As described above, the surgical instrument may obtain information on the operational state of the end tool by sensing changes in a force applied to the target member, or in movement, deformation, damage, or the like of the target member. In some embodiments, since the end tool receives power through a wire, the surgical instrument may derive tension information of the wire based on the sensing data and obtain information on the operational state of the end tool. In this case, the tension information of the wire may be obtained based on a change in a predetermined member, i.e., the target member, that supports a portion of the wire or is connected to the wire to receive a force. Accordingly, the surgical instrument may more accurately obtain information on the operational state of the end tool without directly sensing the operation of the end tool or estimating the operational state of the end tool from the output of the motor or the like.

A surgical instrument according to the present disclosure may generate sensing data by allowing a sensor part to detect a force applied to a predetermined member of a power transmission part or a power generation part, or a movement or deformation of the member, caused by the operation of an end tool. Accordingly, the surgical instrument of the present disclosure may monitor an operational state of the end tool and control the end tool to operate stably within a preset driving range.

A surgical instrument according to an embodiment of the present disclosure can obtain information on an operational state of an end tool by detecting, through a sensor part, physical changes such as a force applied to a target member, or a movement, deformation, or damage of the target member, and control the end tool to operate under preset driving conditions based on the obtained information.

A surgical instrument according to an embodiment of the present disclosure can more accurately and efficiently obtain information on an operational state of an end tool by sensing a change in a target member in a power transmission structure that transmits power to the end tool, without directly sensing an operation of the end tool or estimating the operational state of the end tool based on an output of a motor that provides the power to the end tool.

The present disclosure has been described above with a focus on exemplary embodiments. It will be understood by those skilled in the art that various changes in form and details may be made therein without departing from the essential features of the present disclosure. Therefore, the disclosed embodiments should be considered in descriptive sense only and not for purposes of limitation. The scope of the present disclosure is defined not by the detailed description of the disclosure but by the appended claims, and all differences within the scope will be construed as being included in the present disclosure.

What is claimed is:

1. A surgical instrument comprising:
an end tool having one or more jaws;
a power transmission part including one or more wires connected to the end tool, the power transmission part configured to transmit power generated from a power generation part to the end tool;
a target member provided in one of the power transmission part and the power generation part; and
a sensor part configured to detect a change in the target member according to an operation of the end tool and generate sensing data to obtain information on an operational state of the end tool.

2. The surgical instrument of claim 1, wherein
the target member supports a portion of the one or more wires, and
the surgical instrument further comprises a control part configured to derive tension information of the one or more wires based on the sensing data related to the change in the target member.

3. The surgical instrument of claim 2, wherein the sensor part is configured to obtain the sensing data by detecting a force applied to the target member by the one or more wires.

4. The surgical instrument of claim 2, wherein the sensor part is configured to obtain the sensing data by detecting a movement or a deformation of the target member caused by a force applied by the one or more wires.

5. The surgical instrument of claim 2, wherein the sensor part is disposed between the target member and a support member supporting the target member, and is configured to detect a force applied by the target member to the support member or a relative movement of the target member with respect to the support member.

6. The surgical instrument of claim 5, wherein the sensor part includes a switch member disposed between the target member and the support member, the switch member configured to operate according to a movement or a deformation of the target member.

7. The surgical instrument of claim 6, wherein
the sensor part further includes a connection member disposed between the target member and the support member to be elastically deformable, and
the switch member is configured to operate by receiving an external force when the connection member elastically deforms by at least a predetermined amount during the relative movement of the target member with respect to the support member.

8. The surgical instrument of claim 6, wherein the sensor part is configured to detect a damage of the switch member when a stress greater than or equal to a preset value is applied to the switch member during the movement or the deformation of the target member.

9. The surgical instrument of claim 5, wherein the sensor part is configured to detect contact between the target member and the support member during the relative movement of the target member with respect to the support member.

10. The surgical instrument of claim 1, wherein the power transmission part includes:
a drive pulley around which the one or more wires are wound, the drive pulley configured to rotate around an axis of the drive pulley by receiving the power generated from the power generation part; and
a pulley frame connected to the power generation part and configured to support the drive pulley.

11. The surgical instrument of claim 10, wherein
the power transmission part further includes:
an auxiliary pulley disposed inside the pulley frame and configured to guide a path of the one or more wires; and
an auxiliary pulley support member configured to support the auxiliary pulley,
the target member is the auxiliary pulley, and
the sensor part is disposed between the pulley frame and the auxiliary pulley support member and configured to detect a change in the auxiliary pulley.

12. The surgical instrument of claim 11, wherein the sensor part is configured to obtain the sensing data by detecting a force applied by the auxiliary pulley support member to the pulley frame.

13. The surgical instrument of claim 11, wherein the sensor part is configured to obtain the sensing data by detecting a movement or a deformation of the auxiliary pulley support member relative to the pulley frame.

14. The surgical instrument of claim 10, wherein
the drive pulley has one end inserted into a drive pulley coupling part of the pulley frame,
the target member is the drive pulley, and
the sensor part is disposed between the drive pulley and the drive pulley coupling part and configured to detect a change in the drive pulley.

15. The surgical instrument of claim 14, wherein the sensor part is configured to obtain the sensing data by measuring a force applied by the drive pulley to the pulley frame.

16. The surgical instrument of claim 14, wherein the sensor part is configured to obtain the sensing data by sensing a movement or a deformation of the drive pulley relative to the pulley frame.

17. The surgical instrument of claim 1, wherein
the power transmission part includes a power transmission unit configured to move the one or more wires by a driving of a motor included in the power generation part, and
the sensor part is configured to detect a change in the target member that supports the power transmission unit.

18. The surgical instrument of claim 17, wherein
the power transmission unit includes:
a lead screw configured to rotate by the driving of the motor, the lead screw having threads and one end coupled to a support plate; and
a linear movement guide connected to the one or more wires and threadedly engaged with the lead screw, the linear movement guide configured to linearly move according to a rotation of the lead screw,
the target member is the lead screw, and
the sensor part is configured to obtain the sensing data by measuring a force transmitted to the lead screw by the one or more wires.

19. The surgical instrument of claim 18, wherein the sensor part is configured to:
detect a force applied to the support plate to which the lead screw is coupled, and measure a force transmitted to the lead screw by a linear movement of the one or more wires.

20. The surgical instrument of claim 1, further comprising a control part configured to: derive the information on the operational state of the end tool based on the sensing data obtained by the sensor part, and control the end tool to operate within a preset driving condition.
